(19) United States
(12) Patent Application Publication
(10) Pub. No.: US 2026/0132096 A1
(43) Pub. Date: May 14, 2026

(54) IMPROVED PROCESS FOR PRODUCTION OF ALKALI METAL METHOXIDES

(71) Applicant: Evonik Operations GmbH, Essen (DE)

(72) Inventors: Niklas PAUL, Marl (DE); Moritz SCHRÖDER, Münster (DE); Armin Matthias RIX, Marl (DE); Martin WÜLLER, Marl (DE); Dirk ROETTGER, Köln (DE); Tanita Valèrie SIX, Dortmund (DE); Patrick FRANCOIS, Essen (DE); Min-Zae OH, Singapore (SG); Philp ZITZEWITZ, Haltern am See (DE); Johannes RUWWE, Hanau (DE)

(73) Assignee: Evonik Operations GmbH, Essen (DE)

(21) Appl. No.: 19/121,093

(22) PCT Filed: Oct. 20, 2022
(86) PCT No.: PCT/EP2022/079247

§ 371 Date: Apr. 14, 2025

(51) Int. Cl. C07C 29/70; C07C 29/68

(52) U.S. Cl. CPC C07C 29/70; C07C 29/685

(57) ABSTRACT

The present invention relates to a process for producing at least one alkali metal methoxide by reactive distillation in at least one reaction column. At the lower end of the reaction column(s), the respective alkali metal methoxide dissolved in methanol is withdrawn. The methanol/water mixture obtained at the top of the reaction column(s) is separated by distillation in a rectification column. The energy from the vapour obtained at the top of the rectification column is transferred to a liquid or gaseous heat transfer medium, and the gaseous heat transfer medium obtained as a result is compressed in at least two stages. The energy from the respectively compressed heat transfer medium is advantageously transferred to the bottom stream and sidestream from the rectification column. This enables particularly energy-efficient use of the energy from the vapour in the process of the invention. The energy from the compressed heat transfer medium may additionally be used for operation of the reaction column(s) or for operation of a reaction column in which a process for transalcoholization of alkali metal alkoxides is conducted.

The present invention relates to a process for preparing at least one alkali metal methoxide by reactive distillation in at least one reaction column. At the lower end of the reaction column(s), the respective alkali metal methoxide dissolved in methanol is withdrawn. The methanol/water mixture obtained at the top of the reaction column(s) is separated by distillation in a rectification column. The energy of the vapour obtained at the top of the rectification column is transferred to a liquid or gaseous heat transfer medium, and the gaseous heat transfer medium obtained thereby is compressed in at least two stages. The energy from the respectively compressed heat transfer medium is advantageously transferred to the bottom stream and side stream from the rectification column. This enables particularly energy-efficient use of the energy from the vapour in the process according to the invention.

The energy from the compressed heat transfer medium may additionally be used for operation of the reaction column(s) or for operation of a reaction column in which a process for transalcoholization of alkali metal alkoxides is performed.

### 1. BACKGROUND OF THE INVENTION

Alkali metal alkoxides are used as strong bases in the synthesis of numerous chemicals, for example in the production of pharmaceutical or agrochemical active ingredients. Alkali metal alkoxides are also used as catalysts in transesterification and amidation reactions.

Alkali metal alkoxides (MOR where R is the alkyl radical of the respective alcohol, especially R=C₁ to C₆-alkyl, preferably methyl, ethyl, iso-propyl, n-propyl) are prepared by reactive distillation in a countercurrent distillation column from alkali metal hydroxides (MOH) and alcohols (ROH), with removal of the water of reaction formed according to the following reaction <1> together with the distillate:

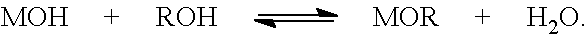

Such a process principle is described, for example, in U.S. Pat. No. 2,877,274 A, wherein aqueous alkali metal hydroxide solution and gaseous methanol are run in countercurrent in a reactive rectification column. This method is described again in basically unchanged form in WO 01/42178 A1.

Methods that are similar, but in which an entraining agent, for example benzene, is additionally used, are described in GB 377,631 A and U.S. Pat. No. 1,910,331 A. This entraining agent is used to separate water and the water-soluble alcohol. In both patent specifications the condensate is subjected to a phase separation to separate off the water of reaction. A further similar process is the reaction of an alkali metal alkoxide with another alcohol in a reaction column (“transalcoholization”) according to DE 27 26 491 A1.

Correspondingly, DE 96 89 03 C describes a method of continuous preparation of alkali metal alkoxides in a reaction column, wherein the water-alcohol mixture withdrawn at the top of the column is condensed and then subjected to a phase separation. The aqueous phase is discarded and the alcoholic phase is returned to the top of the column together with the fresh alcohol. EP 0 299 577 A2 describes a similar method, wherein the water in the condensate is separated off with the aid of a membrane.

The most industrially important alkali metal alkoxides are those of sodium and potassium, especially the methoxides and ethoxides. Their synthesis is frequently described in the prior art, for example in EP 1 997 794 A1.

The syntheses of alkali metal alkoxides by reactive rectification described in the prior art typically afford vapours comprising the alcohol used and water. It is advantageous for economic reasons to reuse the alcohol present in the vapours as a reactant in the reactive distillation. The vapours are therefore typically supplied to a rectification column and the alcohol present therein is separated off (described for example in GB 737 453 A and U.S. Pat. No. 4,566,947 A). The alcohol thus recovered is then fed to the reactive distillation as a reactant for example.

WO 2021/148174 A1 and WO 2021/148175 A1 describe the parallel preparation of different alkali metal alkoxides in separate reaction columns, wherein the vapours obtained in the respective reaction column are separated into the respective alcohol and water in a rectification column.

Alternatively or in addition a portion of the alcohol vapour may be utilized for heating the rectification column (described in WO 2010/097318 A1). However, this requires that the vapour be compressed in order to achieve the temperature level required for heating the rectification column. In particular, a multistage compression of the vapour is thermodynamically advantageous. The vapour is cooled here between the compression stages. The intermediate cooling also ensures that the maximum permissible temperature of the compressor is not exceeded. The disadvantage of this cooling performed in the conventional processes is that the energy thus withdrawn is dissipated without being utilized.

There is accordingly a need for improved processes for workup of an alcohol/water mixture in the context of a process for preparing alkali metal alkoxides, where the alcohol is especially methanol. This process is to feature particularly efficient utilization of the energy present in the vapour for operation of the rectification column.

### 2. BRIEF SUMMARY OF THE INVENTION

The present invention accordingly relates to a process for preparing at least one alkali metal methoxide of the formula M_{A}OCH₃ where M_{A} is selected from sodium, potassium, lithium, especially from sodium, potassium, and is preferably sodium.

Optionally, simultaneously and spatially separately from the conversion to the alkali metal methoxide of the formula M_{A}OCH₃, in a second reaction column RR_{B}, a further alkali metal methoxide of the formula M_{B}OCH₃ is prepared, where M_{B} is selected from sodium, potassium, lithium, especially from sodium, potassium, and is preferably potassium.

This affords, at the top of the reaction column RR_{A} or of the reaction columns RR_{A} and RR_{B}, one vapour stream S_{AB} or two vapour streams S_{AB} and S_{BB}, each comprising water and methanol. Stream S_{AB} or streams S_{AB} and S_{BB} are directed individually (i.e. not mixed with one another) or mixed with one another into a rectification column RD_{A}, where they are separated by distillation into water and methanol. Methanol is obtained at the top of RD_{A} as vapour stream S_{OA}. The energy from S_{OA} is advantageously integrated into the process by means of a heat transfer medium W*1 that functions as working medium. This transfers energy from at least a portion of S_{OA} to the liquid or gaseous heat transfer medium W*₁, in which case, if W*₁ is liquid, the latter is at least partly evaporated. Accordingly, a gaseous heat transfer medium W*₂ is obtained, both when W*₁ is gaseous and when W*₁ is liquid. The gaseous heat transfer medium W*₂ is then at least partly compressed, which affords a gaseous heat transfer medium W*₃ that has been compressed relative to W*₂. W*₃ is divided into at least two portions W*₃₁ and W*₃₂, and energy is transferred from W*₃₁ to a side stream S_{ZA} from the rectification column RD_{A}. W*₃₂ is compressed further, which affords a gaseous heat transfer medium W*₄ that has been compressed relative to W*₃₁. Finally, energy is transferred from W*₄ to a stream S_{UA1} that has been withdrawn from the bottom of RD_{A}, and S_{UA1} is then recycled into RD_{A}.

In a further preferred aspect, the present invention relates to a process for transalcoholization of alkali metal alkoxides. In this process, the alcohol radical of an alkali metal alkoxide M_{c}OR′ is exchanged for another alcohol R″OH, where R′ and R″ are two different C₁ to C₆ hydrocarbon radicals; in particular, R′=methyl and R″=C₂ to C₆ hydrocarbon radical, preferably R′=methyl and R″=ethyl, n-propyl, iso-propyl, more preferably R′=methyl and R″=ethyl.

This involves reacting M_{c}OR′ with R″OH in a reaction column to give M_{c}OR″, and using energy from W*₃, especially W*₃₁ or W*₃₂, or W*₄ in the process for transalcoholization.

### 3. FIGURES

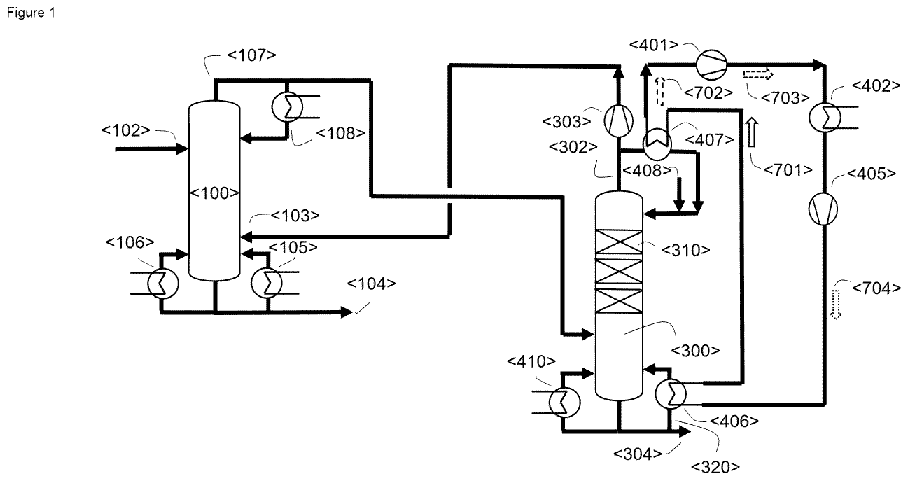

### 3.1 FIG. 1

FIG. 1 shows a comparative process for preparing alkali metal methoxide, in which the distillative separation of the methanol-water mixture is not according to the invention.

This involves reacting aqueous NaOH S_{AE2} <102> in a reaction column RR_{A} <100> with methanol S_{AE1} <103> to give methanolic sodium methoxide solution. At the top of the reaction column RR_{A} <100> an aqueous NaOH solution is added as reactant stream S_{AE2} <102>. It is alternatively also possible to add a methanolic NaOH solution as reactant stream S_{AE2} <102>. The corresponding potassium methoxide is prepared by adding aqueous or methanolic KOH solution as reactant stream S_{AE2} <102>. Above the bottom of the reaction column RR_{A} <100>, methanol is added in vapour form as reactant stream S_{AE1} <103>.

At the bottom of the reaction column RR_{A} <100>, a solution of the corresponding methoxide in methanol S_{AP*} <104> is withdrawn. The bottom evaporator V_{SA} <105> and the optional evaporator V_{SA′} <106> at the bottom of the column RR_{A} <100> are used to adjust the concentration of the sodium methoxide solution S_{AP*} <104> to the desired value.

At the top of the reaction column RR_{A} <100>, a vapour stream S_{AB} <107> is withdrawn. A portion of the vapour stream S_{AB} <107> is condensed in the condenser K_{RRA} <108> and applied in liquid form to the top of the reaction column RR_{A} <100> as reflux. However, condenser K_{RRA} <108> and the establishment of the reflux are optional.

The vapour S_{AB} <107> obtained is sent wholly or partly to a rectification column, the water/methanol column, RD_{A} <300>. The rectification column RD_{A} <300> contains internals <310>. The water/methanol mixture is distillatively separated therein, and methanol is recovered by distillation overhead as vapour S_{OA} <302>.

A reflux is established in the rectification column RD_{A} <300>. A portion of the vapour S_{OA} <302> is condensed in a condenser K_{RD} <407>. The condensation of the stream directed through K_{RD} <407> may be completed in a further condenser beyond K_{RD} <407> with another cooling medium (water, air). The portion of the vapour S_{OA} <302> thus condensed is then recycled back to the rectification column RD_{A} <300>. The remaining portion of S_{OA} <302>, i.e. that not sent to the condenser K_{RD} <407>, is compressed by means of compressor V_{DAB2} <303> and recycled to the reaction column RR_{A} <100>, where it is used as reactant stream S_{AE1} <103>.

In the condenser K_{RD} <407>, energy is transferred from a portion of S_{OA} <302> to a liquid heat transfer medium W*₁ <701>, which is preferably n-butane. W*₁ <701> is evaporated thereby, and a gaseous heat transfer medium W*₂ <702> is obtained. W*₂ <702> is fed to the compressor VD₁ <401>, and W*₂ <702> is optionally additionally heated (not shown in FIG. 1) before being fed to the compressor VD₁ <401>. In VD₁ <401>, W*₂ <702> is compressed further to give the gaseous heat transfer medium stream W*₃ <703>, from which energy can be removed in the optional intermediate cooler WT_{X} <402>.

W*₃ <703> is compressed further by means of compressor VD_{X} <405>, and the resultant gaseous heat transfer medium stream W*₄ <704> is fed to the evaporator V_{SRD} <406> at the bottom of the rectification column RD_{A} <300> for heating. As a result of the release of energy, W*₄ <704> becomes W*₁ <701> again; in particular W*₄ <704> is at least partly condensed, again affording W*₁ <701>, which then undergoes a new cycle again as described above.

Fresh methanol <408> can be fed to the process via the reflux into the rectification column RD_{A} <300>.

Obtained at the bottom of the rectification column RD_{A} <300> is a water stream S_{UA} <304> which is at least partly (stream S_{UA1} <320>) recycled back into the rectification column RD_{A} <300>, in which case it is passed through the evaporator V_{SRD} <406> and/or V_{SRD′} <410>.

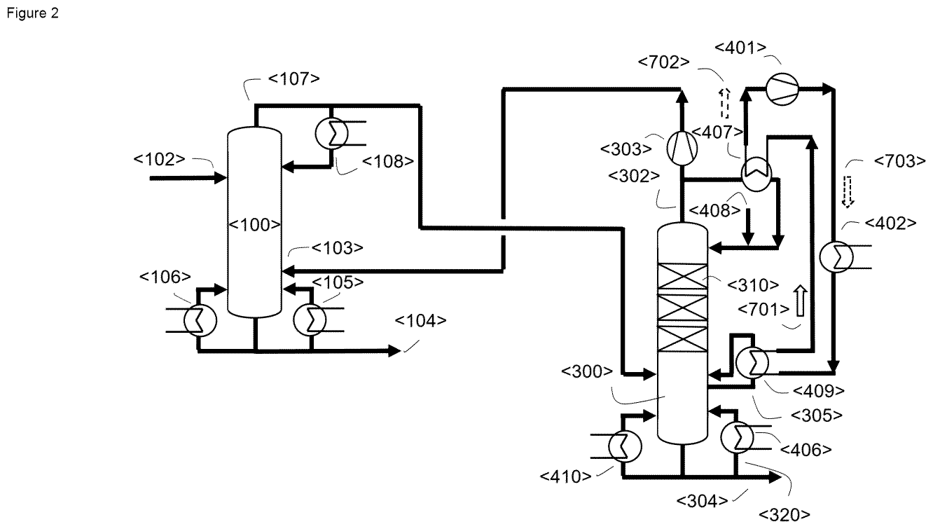

### 3.2 FIG. 2

FIG. 2 shows a further comparative process for preparing alkali metal methoxide, in which the distillative separation of the methanol-water mixture is not according to the invention.

This embodiment corresponds to the one described in FIG. 1 with the following additional/different features: In addition to the evaporators V_{SRD′} <406> and V_{SRD′} <410> at the bottom, the rectification column RD_{A} <300> comprises an intermediate evaporator V_{ZRD} <409>. A side stream S_{ZA} <305> is withdrawn from the rectification column RD_{A} <300> and passed through V_{ZRD} <409> before being returned to the rectification column RD_{A} <300>. A portion of the vapour stream S_{OA} <302> is compressed by means of compressor VD_{AB2} <303> and recycled as reactant stream S_{AE1} <103> to the reaction column RR_{A} <100>.

The other portion of the vapour S_{OA} <302> is condensed in a condenser K_{RD} <407> and then recycled back to the rectification column RD_{A} <300>. The condensation of the stream directed through K_{RD} <407> may be completed in a further condenser beyond K_{RD} <407> with another cooling medium (water, air).

In the condenser K_{RD} <407>, energy is transferred from a portion of S_{OA} <302> to a liquid heat transfer medium W*₁ <701>, which is preferably n-butane. W*₁ <701> is evaporated thereby, and a gaseous heat transfer medium W*₂ <702> is obtained. W*₂ <702> is fed to the compressor VD₁ <401>, where it is compressed further to give the gaseous heat transfer medium stream W*₃ <703>, from which energy can be removed in the optional intermediate cooler WT_{X} <402>.

The gaseous heat transfer medium stream W*₃ <703> is fed to the intermediate evaporator V_{ZRD} <409> for heating. There is no heating in the evaporator V_{SRD} <406> or in the evaporator V_{SRD′} <410> by W*₃ <703>. As a result of the release of energy, W*₃ <703> becomes W*₁ <701> again; in particular W*₃ <703> is at least partly condensed, again affording W*₁ <701>, which then undergoes a new cycle again as described above.

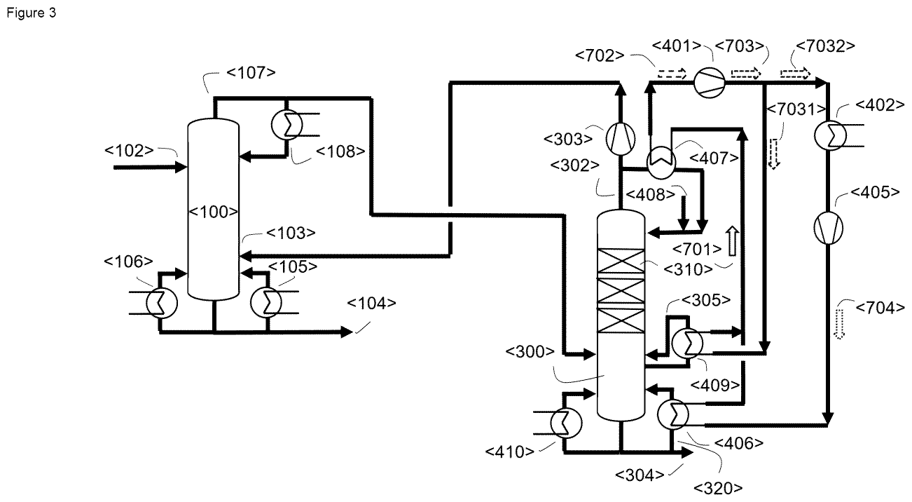

### 3.3 FIG. 3

FIG. 3 shows one embodiment of the process according to the invention. The rectification column RD_{A} <300> used therein comprises an intermediate evaporator V_{ZRD} <409> and a reboiler V_{SRD} <406>, and optionally the reboiler V_{SRD′} <410>.

This embodiment of the invention has the following differences from the embodiments described in FIGS. 1 and 2:
1. After compression of the gaseous heat transfer medium W*₂ <702> in the compressor VD₁ <401>, the gaseous heat transfer medium stream W*₃ <703> is divided into two portions W*₃₁ <7031> and W*₃₂ <7032>.
2. W*₃₁ <7031> is fed to the intermediate evaporator V_{ZRD} <409> for heating of stream S_{ZA} <305>.
3. W*₃₂ <7032> is compressed further in the compressor VDₓ <405> to give stream W*₄ <704>. In an optional embodiment, energy is removed from W*₃₂ <7032> in the optional intermediate cooler WTₓ <402> before W*₃₂ <7032> is compressed. W*₄ <704> is fed to the reboiler V_{SRD} <406> for heating of stream S_{UA1} <320>.
4. Once W*₃₁ <7031> and W*₄ <704> have left the respective evaporator V_{ZRD} <409> or V_{SRD} <406>, and condense especially as a result of the release of energy to the respective stream, they are combined and can undergo a new cycle as W*_{1<701>.}

On account of the differences in the inventive procedure and the division of the compressed gaseous heat transfer medium stream W*₃ <703> into two portions W*₃₁ <7031> and W*₃₂ <7032>, of which only W*₃₂ <7032> is additionally compressed, the energy from the once-compressed stream W*₃₁ <7031> or twice-compressed stream W*₄ <704>, by comparison with the embodiment according to FIGS. 1 and 2, can be utilized more efficiently for heating of the rectification column RD_{A} <300> by means of the intermediate evaporator V_{ZRD} <409> or the reboiler V_{SRD} <406>.

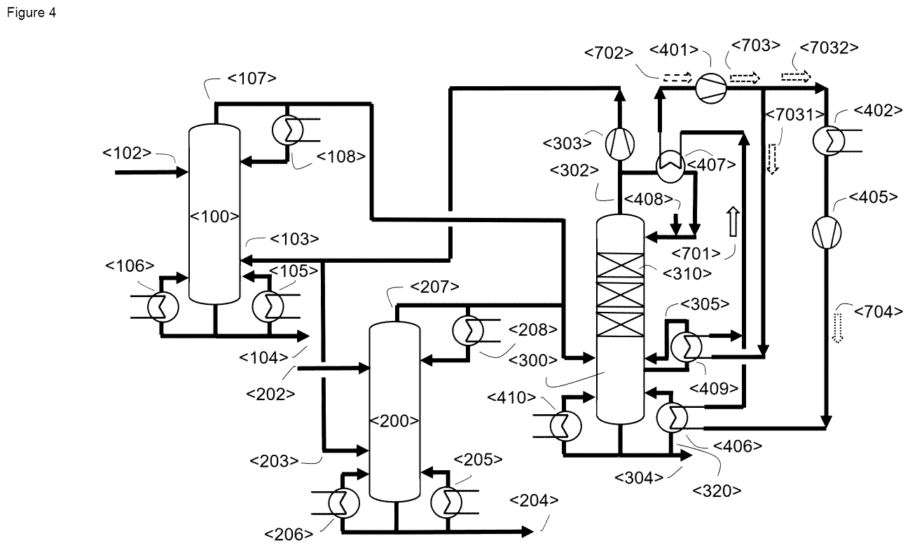

### 3.4 FIG. 4

FIG. 4 shows one embodiment of the process according to the invention. This corresponds to the embodiments described in FIG. 3, with the difference that, in a second reaction column RR_{B}<200>, an aqueous KOH solution S_{BE2} <202> is reacted with methanol S_{BE1} <203> to give potassium methoxide.

At the top of the reaction column RR_{B} <200> an aqueous KOH solution is added as reactant stream S_{BE2} <202>. It is alternatively possible to add a methanolic KOH solution as reactant stream S_{BE2} <202>. Above the bottom of the reaction column RR_{B} <200>, methanol is added in vapour form as reactant stream S_{BE1} <203>.

At the bottom of the reaction column RR_{B} <200>, a mixture of the corresponding methoxide in methanol S_{BP′} <204> is withdrawn. The reboiler V_{SB} <205> and the optional evaporator V_{SB′} <206> at the bottom of the column RR_{B} <200> are used to adjust the concentration of the potassium methoxide solution S_{BP′} <204> to the desired value.

At the top of the reaction column RR_{B} <200>, a vapour stream S_{BB} <207> is withdrawn. A portion of the vapour stream S_{BB} <207> is condensed in the condenser K_{RRB} <208> and applied in liquid form as reflux to the top of the reaction column RR_{B} <200>. However, condenser K_{RRB} <208> and the establishment of the reflux are optional.

The obtained vapour S_{BB} <207> is supplied to the rectification column RD_{A} <300> in admixture with the portion of the vapour S_{AB} <107> not condensed in the condenser K_{RRA} <108>. Alternatively, the vapours S_{AB} <107> and S_{BB} <207> may also be fed to the rectification column RD_{A} <300> separately, i.e. at two different feed points. These two feed points are preferably in the lower half of RD_{A} <300>, preferably beneath the internals <310>.

A further difference from the embodiment according to FIG. 3 is that the portion of the vapour S_{OA} <302> compressed by means of compressor V_{DAB2} <303> is partly recycled to the reaction column RR_{A} <100> and RR_{B} <200>, where it is used as reactant stream S_{AE1} <103>/S_{BE1} <203>.

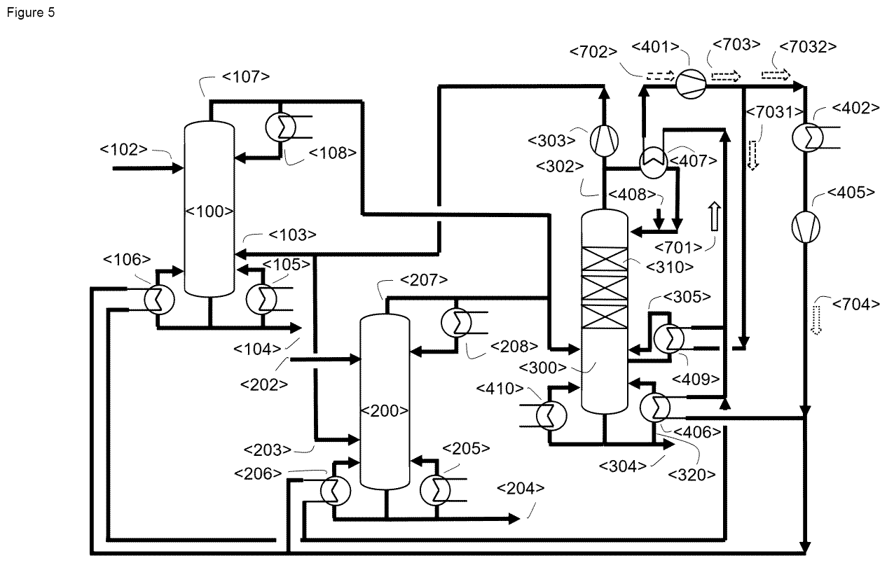

### 3.5 FIG. 5

FIG. 5 shows a further embodiment of the process according to the invention. This corresponds to the embodiments described in FIG. 4, with the difference that a portion of W*₄ <704> is also utilized for heating the evaporator V_{SA′} <106> at the bottom of the column RR_{A} <100> and the evaporator V_{SB′}<206> at the bottom of the column RR_{B} <200>.

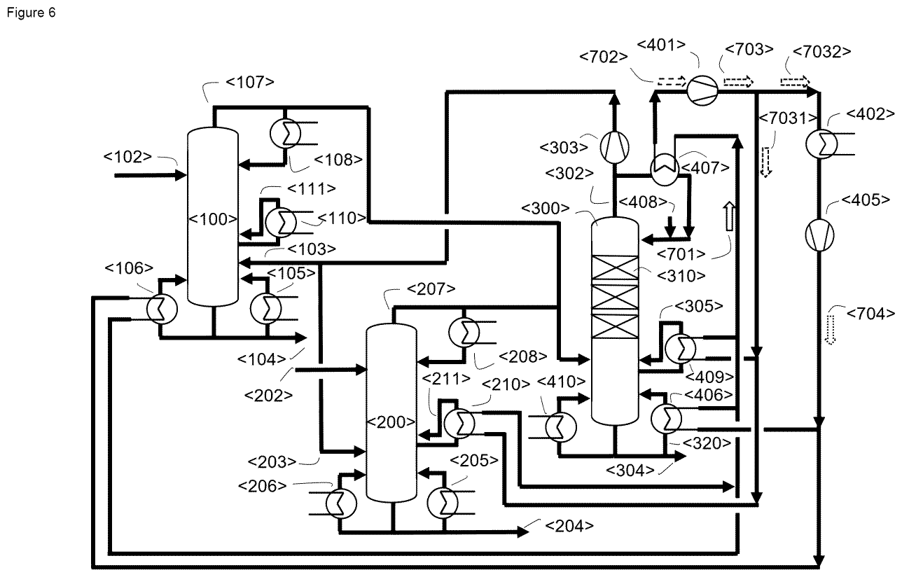

### 3.6 FIG. 6

FIG. 6 shows a further embodiment of the process according to the invention. This corresponds to the embodiment described in FIG. 5 with the difference that the reaction columns RR_{A} <100> and RR_{B} <200> each comprise an intermediate evaporator V_{ZA} <110> and V_{ZB} <210>. A side stream S_{ZAA} <111> is withdrawn from the reaction column RR_{A} <100> and passed through V_{ZA} <110> before being returned to the reaction column RR_{A} <100>. A side stream S_{ZBA} <211> is withdrawn from the reaction column RR_{B} <200> and passed through V_{ZB} <210> before being returned to the reaction column RR_{B} <200>.

By contrast with FIG. 5, a portion of W*₄ <704> is utilized solely for heating of the evaporator V_{SA′} <106> at the bottom of the column RR_{A} <100>, but not for heating of the evaporator V_{SB′} <206> at the bottom of the column RR_{B} <200>. By contrast, a portion of W*₃₁ <7031> is utilized for heating of the evaporator V_{ZB} <210>.

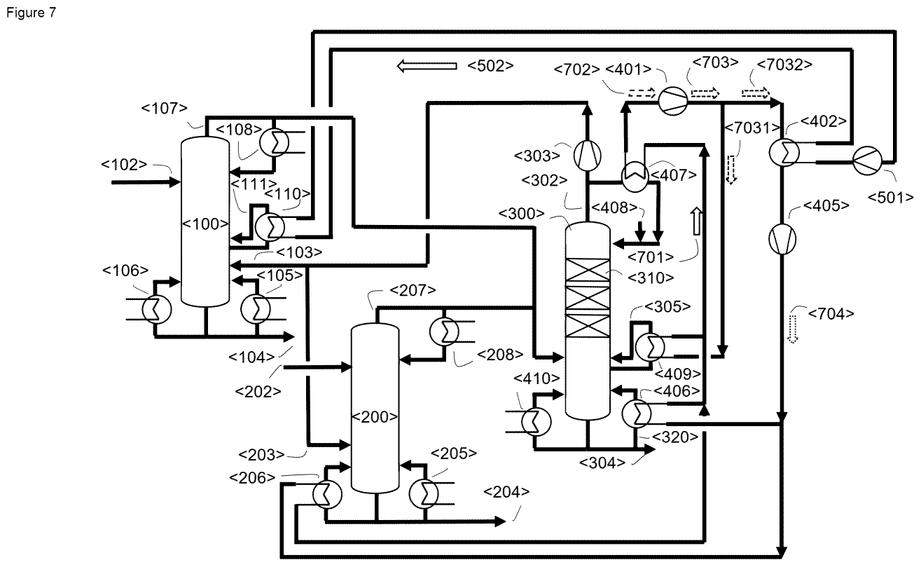

### 3.7 FIG. 7

FIG. 7 shows a further embodiment of the process according to the invention. This corresponds to the embodiments described in FIG. 5 with the difference that the reaction column RR_{A} <100> comprises an intermediate evaporator V_{ZA} <110>. A side stream S_{ZAA} <111> is withdrawn from the reaction column RR_{A} <100> and passed through V_{ZA} <110> before being returned to the reaction column RR_{A} <100>. By contrast with FIG. 5, a portion of W*₄ <704> is utilized solely for heating of the evaporator V_{SB′} <206> at the bottom of the column RR_{B} <200>, but not for heating of the evaporator V_{SA′} <106> at the bottom of the column RR_{A} <100>. The intermediate evaporator V_{ZA} <110> is heated via a heat transfer medium W* <502>, in particular water, which is transported by a pump <501> and which absorbs heat from W*₃₂ <7032> in the intermediate cooler WT_{X} <402> and releases it in the intermediate evaporator V_{ZA} <110>.

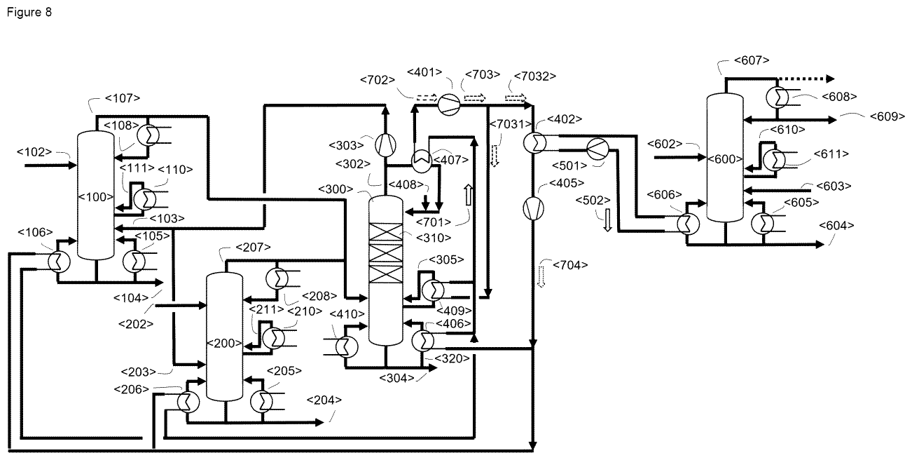

### 3.8 FIG. 8

FIG. 8 shows a further embodiment of the process according to the invention. This corresponds to the embodiment described in FIG. 5 with the difference that the reaction columns RR_{A} <100> and RR_{B} <200> each comprise an intermediate evaporator V_{ZA} <110> and V_{ZB} <210>. A side stream S_{ZAA} <111> is withdrawn from the reaction column RR_{A} <100> and passed through V_{ZA} <110> before being returned to the reaction column RR_{A} <100>. A side stream S_{ZBA} <211> is withdrawn from the reaction column RR_{B} <200> and passed through V_{ZB} <210> before being returned to the reaction column RR_{B} <200>.

In addition, FIG. 8 shows a further preferred embodiment of the process according to the invention. It shows a reactive rectification column RR_{C} <600> for transalcoholization of sodium methoxide to sodium ethoxide which is at least partially operated with energy from stream W*₃₂ <7032>. The column RR_{C} <600> comprises the reboilers V_{SC} <605> and V_{SC} <606>.

This involves reacting sodium methoxide solution S_{CE1} <602> in a reaction column RR_{C} <600> in countercurrent with ethanol S_{CE2} <603> to give sodium ethoxide and this is withdrawn as ethanolic solution S_{CP} <604>.

At the bottom of the reaction column RR_{C} <600>, a bottom product stream S_{CP} <604> comprising sodium ethoxide is accordingly withdrawn.

At the top of the reaction column RR_{C} <600>, a vapour stream S_{CB} <607> is withdrawn. Preferably, at least a portion of the vapour stream S_{CB} <607> is condensed in the condenser K_{RRC} <608>, and at least a portion thereof is applied in liquid form to the top of the reaction column RR_{C} <600> as reflux. The vapour stream S_{CB} <607> is partly withdrawn either in gaseous form upstream of the condenser K_{RRC} <608> (marked by dashed line) and/or partly in liquid form downstream of the condenser K_{RRC} <608> as stream <609>.

A side stream S_{ZC} <610> is preferably withdrawn from the reaction column RR_{C} <600>, with transfer of energy to said stream via an intermediate evaporator V_{ZC} <611>, and S_{ZC} <610> may then be recycled into RR_{C} <600>.

As the sodium methoxide solution S_{CE1} <602> it is preferable to utilize at least a portion of the bottom streams S_{AP′} <104> and S_{BP′} <204> obtained in the reaction columns RR_{A} <100> and RR_{B}<200>.

The reboiler V_{SC′}<606> is heated via a heat transfer medium W* <502>, in particular water, which is transported by a pump <501> and which absorbs heat from W*₃₂ <7032> in the intermediate cooler WT_{X} <402> and releases it in the reboiler V_{SC} <606>.

Alternatively, energy may also be appropriately transferred to the reboiler V_{SC} <606> or the other reboiler V_{SC} <605> from another stream selected from W*₄ <704>, W*₃₁ <7031>, W*₃ <703> before the separation into W*₃₁ <7031> and W*₃₂ <7032>. Energy may likewise be transferred to the ethanol stream S_{CE1} <603>, the sodium methoxide solution S_{CE1} <602> or the side stream S_{ZC} <610> from at least one of the streams W*₃ <703>, W*₃₁ <7031>, W*₃₂ <7032>, W*₄ <704>.

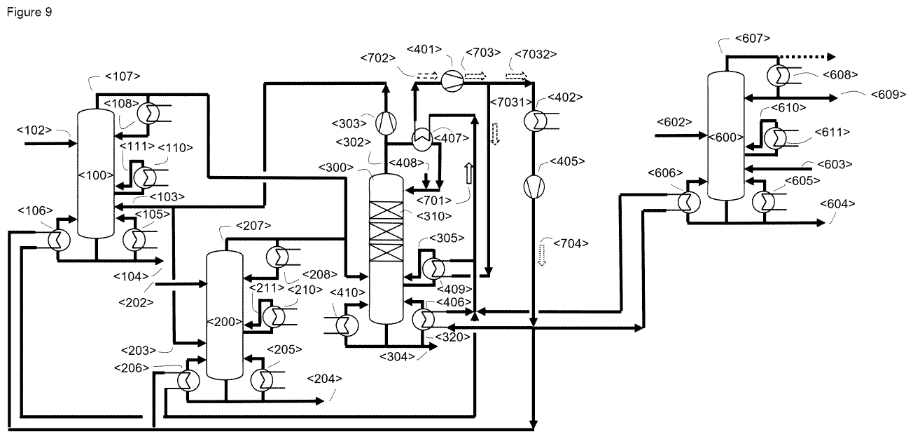

### 3.9 FIG. 9

FIG. 9 shows one embodiment of the process according to the invention. This corresponds to the embodiment described in FIG. 8, with the difference that the reboiler V_{SC} <606> is heated directly with a portion of W*₄ <704>.

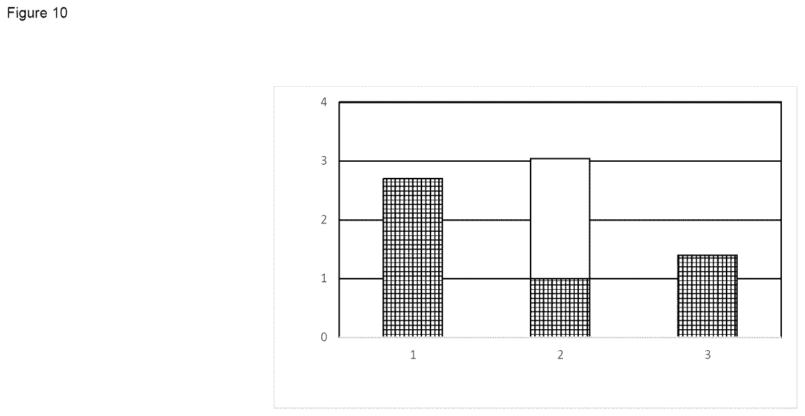

### 3.10 FIG. 10

FIG. 10 illustrates the energy saving in the inventive process according to Example 3 compared to the noninventive process according to Examples 1 and 2. The x axis denotes the respective example, the y axis the power to be applied in MW.

The hatched portion of the bars indicates the sum total of the compressor outputs. The white portion of the bars shows the necessary heating output via low-pressure steam.

### 4. DETAILED DESCRIPTION OF THE INVENTION

The present invention relates to a process for preparing at least one alkali metal methoxide of the formula M_{A}OCH₃ where M_{A} is selected from sodium, potassium, lithium, preferably sodium, potassium, and M_{A} is most preferably sodium.

The process according to the invention is conducted in at least one reactive rectification column, and the vapour streams obtained in the at least one reactive rectification column that comprise methanol and water are then separated in a reaction column at least partly into water and methanol. In this distillative separation, there is efficient integration of the energy of the vapours obtained.

### 4.1 Step (a1)

In step (a1) of the process according to the invention, a reactant stream S_{AE1} comprising methanol is reacted with a reactant stream S_{AE2} comprising M_{A}OH in countercurrent in a reactive rectification column RR_{A} to afford a crude product RP_{A} comprising M_{A}OCH₃, water, methanol, M_{A}OH.

According to the invention a “reactive rectification column” is defined as a rectification column in which, at least in some parts, the reaction according to step (a1) or step (a2) of the process according to the invention proceeds. It may also be abbreviated to “reaction column”.

In step (a1), a bottom product stream S_{AP} comprising methanol and M_{A}OCH₃ is withdrawn at the lower end of RR_{A}. A vapour stream S_{AB} comprising water and methanol is withdrawn at the upper end of RR_{A}.

M_{A} is selected from sodium, lithium, potassium. M_{A} is especially selected from sodium, potassium. Preferably, M_{A}=sodium.

The reactant stream S_{AE1} comprises methanol. In a preferred embodiment, the proportion by mass of methanol in S_{AE1} is ≥95% by weight, yet more preferably ≥99% by weight, and S_{AE1} otherwise comprises especially water.

The methanol used in step (a1) as reactant stream S_{AE1} may also be commercially available methanol having a proportion by mass of methanol of more than 99.8% by weight and a proportion by mass of water of up to 0.2% by weight.

The reactant stream S_{AE1} is preferably introduced in vapour form.

The reactant stream S_{AE2} comprises M_{A}OH. In a preferred embodiment, S_{AE2} comprises not only M_{A}OH but also at least one further compound selected from water, methanol. S_{AE2} more preferably comprises water in addition to M_{A}OH, in which case S_{AE2} is an aqueous solution of M_{A}OH.

When the reactant stream S_{AE2} comprises M_{A}OH and water, the proportion by mass of M_{A}OH, based on the total weight of the aqueous solution forming S_{AE2}, is especially within a range from 10% to 75% by weight, preferably from 15% to 54% by weight, more preferably from 30% to 53% by weight and especially preferably from 40% to 52% by weight.

When the reactant stream S_{AE2} comprises M_{A}OH and methanol, the proportion by mass of M_{A}OH in methanol, based on the total weight of the solution forming S_{AE2}, is especially within a range from 10% to 75% by weight, preferably from 15% to 54% by weight, more preferably from 30% to 53% by weight and especially preferably from 40% to 52% by weight.

In the particular case in which the reactant stream S_{AE2} comprises both water and methanol in addition to M_{A}OH, it is particularly preferable that the proportion by mass of M_{A}OH in methanol and water, based on the total weight of the solution forming S_{AE2}, is especially within a range from 10% to 75% by weight, preferably from 15% to 54% by weight, more preferably from 30% to 53% by weight and particularly preferably from 40% to 52% by weight.

Step (a1) is performed in a reactive rectification column (or “reaction column”) RR_{A}.

Step (a2), elucidated further down, is performed in a reactive rectification column (or “reaction column”) RR_{B}.

The reaction column RR_{A}/RR_{B} preferably contains internals. Suitable internals are, for example, trays, structured packings or unstructured packings. When the reaction column RR_{A}/RR_{B} contains trays, then bubble cap trays, valve trays, tunnel-cap trays, Thormann trays, cross-slit bubble cap trays or sieve trays are suitable. When the reaction column RR_{A}/RR_{B} contains trays it is preferable to choose trays where not more than 5% by weight, more preferably less than 1% by weight, of the liquid trickles through the respective trays. The construction measures required to minimize trickle-through of the liquid are familiar to those skilled in the art. In the case of valve trays, for example, particularly tightly closing valve designs are selected. Reducing the number of valves also makes it possible to increase the vapour velocity in the tray openings to twice the value typically established. When sieve trays are used, it is particularly favourable to reduce the diameters of the tray openings and to maintain or even increase the number of openings.

When structured or unstructured packings are used, structured packings are preferred in terms of uniform distribution of the liquid.

For columns comprising unstructured packings, especially comprising random packings, and for columns comprising structured packings, the desired characteristics of the liquid distribution may be achieved when the liquid trickling density in the edge region of the column cross section adjacent to the column shell, corresponding to about 2% to 5% of the total column cross section, is reduced compared to the other cross-sectional regions by up to 100%, preferably by 5% to 15%. This can easily be achieved by, for example, targeted distributions of the drip points of the liquid distributors or the holes thereof.

The process according to the invention can be performed either continuously or batchwise. It is preferably performed continuously.

According to the invention, the “reaction of a reactant stream S_{AE1} comprising methanol with a reactant stream S_{AE2} comprising M_{A}OH in countercurrent” is achieved more particularly by virtue of the feed point for at least a portion of the reactant stream S_{AE1} comprising methanol in step (a1) being below the feed point of the reactant stream S_{AE2} comprising M_{A}OH in the reaction column RR_{A}.

The reaction column RR_{A} preferably comprises at least 2, in particular 15 to 40, theoretical plates between the feed point of the reactant stream S_{AE1} and the feed point of the reactant stream S_{AE2}.

The reaction column RR_{A} may be operated as a pure stripping column. In that case, the reactant stream S_{AE1} comprising methanol is introduced in vapour form in the lower region of the reaction column RR_{A}.

Step (a1) also encompasses the case where a portion of the reactant stream S_{AE1} comprising methanol is added in vapour form below the feed point of the reactant stream S_{AE2} comprising M_{A}OH but nevertheless at the upper end or in the region of the upper end of the reaction column RR_{A}. This makes it possible to reduce the dimensions of the lower region of the reaction column RR_{A}. When a portion of the reactant stream S_{AE1} comprising methanol is added, especially in vapour form, at the upper end or in the region of the upper end of the reaction column RR_{A}, only a fraction of 10% to 70% by weight, preferably of 30% to 50% by weight, (based in each case on the total amount of methanol used in step (a1)) is introduced at the lower end of the reaction column RR_{A}, and the remaining fraction is added in vapour form in a single stream or divided into a plurality of substreams, preferably 1 to 10 theoretical plates, more preferably 1 to 3 theoretical plates, below the feed point of the reactant stream S_{AE2} comprising M_{A}OH.

In the reaction column RR_{A}, the reactant stream S_{AE1} comprising methanol is then reacted with the reactant stream S_{AE2} comprising M_{A}OH according to the above-described reaction <1> to give M_{A}OCH₃ and H₂O, where these products are present in admixture with the methanol and M_{A}OH reactants since the reaction is an equilibrium reaction. Accordingly, in step (a1), a crude product RP_{A} comprising methanol and M_{A}OH in addition to the M_{A}OCH₃ and water products is obtained in the reaction column RR_{A}.

The bottom product stream S_{AP} comprising methanol and M_{A}OCH₃ is then obtained and withdrawn at the lower end of RR_{A}.

The stream of methanol that still contains water, referred to above as “vapour stream S_{AB} comprising water and methanol”, is withdrawn at the upper end of RR_{A}, preferably at the column top of RR_{A}.

This vapour stream S_{AB} comprising water and methanol is directed in step (a3) at least partly into a rectification column RD_{A}, where it is separated by distillation at least partly into a vapour stream S_{OA} comprising methanol, which is withdrawn at the upper end of RD_{A}, and at least one stream S_{UA} comprising water, which is withdrawn at the lower end of RD_{A}. In the embodiments of the present invention in which step (a2) is conducted, at least a portion of vapour stream S_{BB}, mixed with S_{AB} or separately from S_{AB}, is additionally directed into the rectification column RD_{A}.

A portion of the methanol obtained in stream S_{OA} in the distillation in step (a3) may be fed to the reaction column RR_{A} as reactant stream S_{AE1}.

In a preferred embodiment of the process according to the invention, a portion of S_{OA} is used as reactant stream S_{AE1} in step (a1) and, if step (a2) is performed, alternatively or additionally as reactant stream S_{BE1} in step (a2).

In a more preferred embodiment of the process according to the invention, 5% to 95% by weight, preferably 10% to 90% by weight, more preferably 20% to 80% by weight, yet more preferably 30% to 70% by weight, yet more preferably 50% to 60% by weight, yet more preferably 56.7% by weight, of the vapour stream S_{OA} is used as reactant stream S_{AE1} or, if step (a2) is performed, alternatively or additionally as reactant stream S_{BE1} in step (a2).

In this preferred embodiment, it is advantageous to compress the portion of the stream S_{OA} used as reactant stream S_{AE1}/as reactant stream S_{BE1}.

The amount of methanol encompassed by the reactant stream S_{AE1} is preferably chosen such that it simultaneously serves as solvent for the alkali metal methoxide M_{A}OCH₃ obtained in the bottom product stream S_{AP}. The amount of methanol in the reactant stream S_{AE1} is preferably chosen so as to achieve, in the bottom of the reaction column, the desired concentration of the alkali metal methoxide solution which is withdrawn as bottom product stream S_{AP} comprising methanol and M_{A}OCH₃.

In a preferred embodiment of the process according to the invention, and especially in the cases in which S_{AE2} comprises not only M_{A}OH but also water, the ratio of the total weight (mass; unit: kg) of methanol used as reactant stream S_{AE1} in step (a1) to the total weight (mass; unit: kg) of M_{A}OH used as reactant stream S_{AE2} in step (a1) is 4:1 to 50:1, more preferably 8:1 to 48:1, even more preferably 10:1 to 45:1, yet more preferably 20:1 to 40:1, even more preferably 22:1.

The reaction column RR_{A} is operated with or without, preferably with, reflux.

“With reflux” means that the vapour stream S_{AB}/S_{BB} comprising water and methanol withdrawn at the upper end of the respective column, in step (a1) from the reaction column RR_{A}, in the optional step (a2) from the reaction column RR_{B}, is not completely discharged. In that case, in step (a3), the respective vapour stream S_{AB}/S_{BB} is thus not directed in its entirety into a rectification column RD_{A}, but rather at least partly, preferably partly, returned to the respective column as reflux, in step (a1) to the reaction column RR_{A}, and in the optional step (a2) to the reaction column RR_{B}. In the cases where such a reflux is established, the reflux ratio is preferably 0.01 to 1, more preferably 0.02 to 0.9, yet more preferably 0.03 to 0.34, yet more preferably 0.04 to 0.27, yet more preferably 0.05 to 0.24, yet more preferably 0.06 to 0.10, yet more preferably 0.07 to 0.08.

A reflux can be established by mounting a condenser at the top of the respective column. In step (a1) this is achieved in particular by attaching a condenser K_{RRA} to the reaction column RR_{A}. In step (a2) this is achieved in particular by attaching a condenser K_{RRB} to the reaction column RR_{B}. In the respective condenser, the respective vapour stream S_{AB}/S_{BB} is at least partly condensed and returned to the respective column, in step (a1) to the reaction column RR_{A}, or in step (a2) to the reaction column RR_{B}.

In the embodiment in which a reflux is established in the reaction column RR_{A}, the M_{A}OH used as reactant stream S_{AE2} in step (a1) may also be at least partly mixed with the reflux stream, and the resulting mixture may be supplied as such to step (a1).

Step (a1) is performed especially at a temperature within a range from 45° C. to 150° C., preferably 47° C. to 120° C., more preferably 60° C. to 110° C., and at a pressure of 0.5 bar abs. to 40 bar abs., preferably within a range from 0.7 bar abs. to 5 bar abs., more preferably within a range from 0.8 bar abs. to 4 bar abs., more preferably within a range from 0.9 bar abs. to 3.5 bar abs., yet more preferably at 1.0 bar abs. to 3 bar abs., yet more preferably 1.25 bar abs.

In a preferred embodiment, the reaction column RR_{A} comprises at least one evaporator which is especially selected from intermediate evaporators V_{ZA} and reboilers V_{SA}. The reaction column RR_{A} more preferably comprises at least one bottom evaporator V_{SA}.

According to the invention, “intermediate evaporators” V_{Z} refer to evaporators disposed above the bottom of the respective column, especially above the bottom of the reaction column RR_{A}/RR_{B} (in that case referred to as “V_{ZA}”/“V_{ZB}”) or above the bottom of the rectification column RD_{A} (in that case referred to as “V_{ZRD}”). In the case of RR_{A}/RR_{B}, what is evaporated therein is especially crude product RP_{A}/RP_{B} which is withdrawn from the column as side stream S_{ZAA}/S_{ZBA}.

According to the invention, “reboilers” V_{Z} refer to evaporators which heat the bottom of the respective column, especially the bottom of the reaction column RR_{A}/RR_{B}, or RR_{C} as used in the preferred embodiment and more particularly described hereinafter (in that case referred to as “V_{SA}” or “V_{SA′}”/“V_{SB}” or “V_{SB′}”/“V_{SC}” or “V_{SC′}”) or the bottom of the rectification column RD_{A} (in that case referred to as “V_{SRD}” or “V_{SRD′}”). In the case of RR_{A}/RR_{B}, what is evaporated therein is especially at least a portion of the bottom product stream S_{AP}/S_{BP}. In the case of RR_{C}, what is evaporated therein is especially bottom product stream S_{CP}. In the case of RD_{A}, what is evaporated therein is especially bottom product stream S_{UA} or a portion of S_{UA}, S_{UA1}.

An evaporator is typically disposed outside the respective reaction column or rectification column. Since evaporators transfer energy, in particular heat, from one stream to another, they are heat transferrers WT. The mixture to be evaporated is withdrawn via a takeoff from the column and supplied to the at least one evaporator. In the case of the reaction column RR_{A}/RR_{B}, intermediate evaporation of the crude product RP_{A}/RP_{B} involves drawing it off and supplying it to the at least one intermediate evaporator V_{ZA}/V_{ZB}.

In the case of the rectification column RD_{A}, intermediate evaporation involves withdrawing (“drawing off”) at least one side stream S_{ZA} from RD_{A} and supplying it to the at least one intermediate evaporator V_{ZRD}.

In the case of the rectification column RD_{A}, bottoms evaporation involves withdrawing (“drawing off”) at least one stream S_{UA} from RD_{A} and supplying at least a portion, preferably a portion, thereof to the at least one reboiler V_{SRD}.

The evaporated mixture is recycled back into the respective column optionally with a residual proportion of liquid via at least one feed. When the evaporator is an intermediate evaporator, i.e. in particular an intermediate evaporator V_{ZA}/V_{ZB}/V_{ZRD}, the takeoff via which the respective mixture is withdrawn and supplied to the evaporator is a side stream takeoff, and the feed via which the evaporated mixture is returned to the respective column is a side stream feed. When the evaporator is a reboiler, i.e. heats the column bottoms, i.e. is in particular a reboiler V_{SA}/V_{SB}/V_{SRD}, at least a portion of the bottoms takeoff stream, in particular S_{AP}/S_{BP}, is supplied to the reboiler, evaporated and recycled back into the respective column in the region of the column bottom. However, it is alternatively also possible to form tubes, for example on a suitable tray when using an intermediate evaporator or in the bottom of the respective column, that are traversed by the heat transfer medium, for example the respective compressed heat transfer medium W*₃₁/W*₄ (when V_{S}/V_{Z} is present in the rectification column RD_{A}) or a heat transfer medium W₁. In this case, the evaporation occurs on the tray or in the bottom region of the column. However, it is preferable to arrange the evaporator outside the respective column.

Suitable evaporators that can be used as intermediate evaporators and bottom evaporators include, for example, natural circulation evaporators, forced circulation evaporators, forced circulation flash evaporators, kettle evaporators, falling-film evaporators or thin-film evaporators. Heat exchangers for the evaporator typically that are used in the case of natural circulation evaporators and forced circulation evaporators are a shell-and-tube or plate apparatus. When a shell-and-tube exchanger is used, the heat transfer medium, for example the compressed heat transfer medium W*₃₁/W*₄ in V_{ZRD} or V_{SRD} at the rectification column RD_{A}, or the heat transfer medium W₁ may either flow through the tubes and the mixture to be evaporated around the tubes, or else the heat transfer medium, for example the compressed heat transfer medium W*₃₁/W*₄ in V_{ZRD}/V_{SRD} at the rectification column RD_{A}, or the heat transfer medium W₁ flows around the tubes and the mixture to be evaporated through the tubes. In the case of a falling-film evaporator, the mixture to be evaporated is typically introduced as a thin film on the inside of a tube and the tube is heated externally. In contrast to a falling-film evaporator, a thin-film evaporator additionally comprises a rotor with wipers which distributes the liquid to be evaporated on the inner wall of the tube to form a thin film.

As well as those mentioned, it is also possible to use any desired further evaporator type which is known to those skilled in the art and is suitable for use in a rectification column.

When the evaporator operated, for example, with the compressed heat transfer medium W*₃₁/the heat transfer medium W₁ as heating steam is an intermediate evaporator, it is preferable when the intermediate evaporator is disposed in the stripping section of the rectification column RD_{A} in the region between the feed point(s) of the vapour stream S_{AB}/vapour stream S_{BB} and above the column bottom or, in the case of reaction columns RR_{A}/RR_{B}, below the feed point of the reactant stream S_{AE2}/S_{BE2}. This makes it possible to introduce a predominant proportion of the heat energy via the intermediate evaporator. It is thus possible, for example, to introduce more than 80% of the energy via the intermediate evaporator. According to the invention, the intermediate evaporator is preferably arranged and/or configured such that it is used to introduce more than 10%, in particular more than 20%, of the total energy required for the distillation.

Where an intermediate evaporator is used, it is especially advantageous when the intermediate evaporator is arranged such that the respective rectification column/reaction column has 1 to 50 theoretical plates below the intermediate evaporator and 1 to 200 theoretical plates above the intermediate evaporator. In particular, it is preferable when the rectification column/reaction column has 2 to 10 theoretical plates below the intermediate evaporator and 20 to 80 theoretical plates above the intermediate evaporator.

The side stream takeoff via which the mixture from the rectification column/reaction column is supplied to the intermediate evaporator V_{Z} and the side stream feed via which the evaporated mixture from the intermediate evaporator V_{Z} is returned to the respective rectification column/reaction column may be positioned between the same trays of the column. However, it is also possible for the side stream takeoff and side stream feed to be at different heights.

Such an intermediate evaporator V_{ZA} can convert liquid crude product RP_{A} present in the reaction column RR_{A} and comprising M_{A}OCH₃, water, methanol, M_{A}OH to the gaseous state, thus improving the efficiency of the reaction in step (a1) of the process according to the invention.

Such an intermediate evaporator V_{ZB} can convert liquid crude product RP_{B} present in the reaction column RR_{B} and comprising M_{B}OCH₃, water, methanol, M_{B}OH to the gaseous state, thus improving the efficiency of the reaction in step (a2) of the process according to the invention.

By virtue of the arrangement of one or more intermediate evaporators V_{ZA} in the upper region of the reaction column RR_{A}, it is possible to reduce the dimensions in the lower region of the reaction column RR_{A}. In the embodiment having at least one, preferably two or more, intermediate evaporators V_{ZA}, it is also possible to introduce substreams of the methanol in liquid form in the upper region of the reaction column RR_{A}.

In a further preferred embodiment, energy, preferably heat, is transferred from at least a portion of a heat transfer medium, where the heat transfer medium is selected from W*₂, W*₃, W*₄, preferably from W*₃, W*₄, more preferably W*₃, yet more preferably selected from W*₃₁, W*₃₂, yet more preferably W*₃₂, to the crude product RP_{A} and, if step (a2) is conducted, alternatively or additionally to the crude product RP_{B}. The at least a portion of W*₃ is selected here especially from W*₃₁, W*₃₂.

“Transfer of energy, preferably heat, from at least a portion of a heat transfer medium, where the heat transfer medium is selected from W*₂, W*₃, W*₄, to the crude product RP_{A} and, if step (a2) is conducted, alternatively or additionally to the crude product RP_{B}” accordingly also encompasses the transfer of energy, preferably heat, from at least one heat transfer medium selected from W*₃₁, W*₃₂, or from the heat transfer medium W*₃ before it is separated into W*₃₁, W*₃₂, to the crude product RP_{A} and, if step (a2) is conducted, alternatively or additionally to the crude product RP_{B}. It also encompasses the transfer of energy from a portion of W*₃₁, W*₃₂ to the crude product RP_{A} and, if step (a2) is conducted, alternatively or additionally to the crude product RP_{B}.

For this purpose, in particular, a portion of the respective heat transfer medium selected from W*₂, W*₃, W*₄ is conducted at least partly via an intermediate evaporator V_{ZA}/V_{ZB}, and the energy is transferred from the respective heat transfer medium selected from W*₂, W*₃, W*₄ to the crude product stream drawn off by side draw from RR_{A}/RR_{B}, especially in that the respective heat transfer medium selected from W*₂, W*₃, W*₄ is utilized for heating of the evaporator V_{ZA}/V_{ZB}. Optionally, in this embodiment, a heat transfer medium [symbol] other than W*₂, W*₃, W*₄ is “intermediately inserted”. This means that, at first, energy, especially heat, is transferred from at least one heat transfer medium selected from W*₂, W*₃, W*₄ to [symbol] , and then the energy is transferred from [symbol] to the crude product stream drawn off by side draw from RR_{A}/RR_{B}, especially in that [symbol] is utilized for heating of the evaporator V_{ZA}/V_{ZB}.

Heat transfer media [symbol] utilized may be any of the heat transfer media known to the person skilled in the art; preferably, [symbol] is selected from the group consisting of air, water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium [symbol] used is water or air, more preferably water.

According to the invention, reboilers are disposed at the bottom of the respective rectification column RD_{A}/reaction column RR_{A}/RR_{B}/RR_{c} and are then referred to as “V_{SRD}” or “V_{SRD′}”/“V_{SA}” or “V_{SA}”/“V_{SB}” or “V_{SB}” “V_{SC}” or “V_{SC′}”. A bottom product stream (in particular S_{AP}/S_{BP}) present in the respective column (in particular reaction column RR_{A}/RR_{B}) may be directed into such a reboiler and, for example, methanol may be at least partly removed therefrom. In the case of S_{AP}/S_{BP}, this may afford a bottom product stream S_{AP*} having an elevated proportion by mass of M_{A}OCH₃ compared to S_{AP} or a bottom product stream S_{BP*} having an elevated proportion by mass of M_{B}OCH₃ compared to S_{BP}.

In step (a1) of the process according to the invention, a bottom product stream S_{AP} comprising methanol and M_{A}OCH₃ is withdrawn at the lower end of the reaction column RR_{A}.

It is preferable that the reaction column RR_{A} comprises at least one reboiler V_{SA} through which some of the bottom product stream S_{AP} is then directed and methanol is partly removed therefrom, which affords a bottom product stream S_{AP*} having an elevated proportion by mass of M_{A}OCH₃ compared to S_{AP}.

In another preferred embodiment, therefore, the procedure for transferring energy, preferably heat, from at least a portion of a heat transfer medium, where the heat transfer medium is selected from W*₂, W*₃, W*₄, preferably from W*₃, W*₄, and is more preferably W*₄, to the crude product RP_{A} and, if step (a2) is conducted, alternatively or additionally to the crude product RP_{B}, is as follows:

In that case, in particular, a portion of the respective heat transfer medium selected from W*₂, W*₃, W*₄ is directed at least partly through a reboiler V_{SA}/V_{SB} and the energy is transferred from the respective heat transfer medium selected from W*₂, W*₃, W*₄ to the bottom product stream S_{AP}/S_{BP}, especially in that the respective heat transfer medium selected from W*₂, W*₃, W*₄ is utilized for heating of the evaporator V_{SA}/V_{SB}.

The proportion by mass of M_{A}OCH₃ in the bottom product stream S_{AP*} is elevated in particular compared to the proportion by mass of M_{A}OCH₃ in the bottom product stream S_{AP} by at least 0.5%, preferably by ≥1%, more preferably by ≥2%, yet more preferably by ≥5%.

It is preferable when S_{AP} or, if at least one reboiler V_{SA} through which the bottom product stream S_{AP} is at least partly directed and methanol is at least partly removed therefrom is used, S_{AP*} has a proportion by mass of M_{A}OCH₃ in methanol within a range from 1% to 50% by weight, preferably 5% to 35% by weight, more preferably 15% to 35% by weight, most preferably 20% to 35% by weight, based in each case on the total mass of S_{AP}/S_{AP*}.

The proportion by mass of residual water in S_{AP}/S_{AP*}is preferably <1% by weight, preferably <0.8% by weight, more preferably <0.5% by weight, based on the total mass of S_{AP}/S_{AP*}.

The proportion by mass of reactant M_{A}OH in S_{AP}/S_{AP*} is preferably <1% by weight, preferably <0.8% by weight, more preferably <0.5% by weight, based on the total mass of S_{AP}/S_{AP*}.

### 4.2 Step (a2) (Optional)

Step (a2) is an optional embodiment of the process according to the invention. This means that, in the context of the preferred embodiment of the process according to the invention, step (a2) is or is not performed.

In optional step (a2), simultaneously with and spatially separate from step (a1), a reactant stream S_{BE1} comprising methanol is reacted with a reactant stream S_{BE2} comprising M_{B}OH in countercurrent in a reactive rectification column RR_{B} to afford a crude product RP_{B} comprising M_{B}OCH₃, water, methanol, M_{B}OH.

In optional step (a2) of the process according to the invention, a bottom product stream S_{BP} comprising methanol and M_{B}OCH₃ is withdrawn at the lower end of RR_{B}. A vapour stream S_{BB} comprising water and methanol is withdrawn at the upper end of RR_{B}.

M_{B} is selected from sodium, lithium, potassium. M_{B} is especially selected from sodium, potassium. Preferably, M_{B}=potassium.

The reactant stream S_{BE1} comprises methanol. In a preferred embodiment, the proportion by mass of methanol in S_{BE1} is ≥95% by weight, yet more preferably ≥99% by weight, and S_{BE1} otherwise comprises especially water.

The methanol used in the optional step (a2) of the process according to the invention as reactant stream S_{BE1} may also be commercially available methanol having a proportion by mass of methanol of more than 99.8% by weight and a proportion by mass of water of up to 0.2% by weight.

The reactant stream S_{BE1} is preferably introduced in vapour form.

The reactant stream S_{BE2} comprises M_{B}OH. In a preferred embodiment, S_{BE2} comprises not only M_{B}OH but also at least one further compound selected from water, methanol. It is yet more preferable when S_{BE2} also comprises water in addition to M_{B}OH; in that case, S_{BE2} is an aqueous solution of M_{B}OH.

When the reactant stream S_{BE2} comprises M_{B}OH and water, the proportion by mass of M_{B}OH, based on the total weight of the aqueous solution forming S_{BE2}, is especially within a range from 10% to 75% by weight, preferably from 15% to 54% by weight, more preferably from 30% to 53% by weight and especially preferably from 40% to 52% by weight.

When the reactant stream S_{BE2} comprises M_{B}OH and methanol, the proportion by mass of M_{B}OH in methanol, based on the total weight of the solution forming S_{BE2}, is especially within a range from 10% to 75% by weight, preferably from 15% to 54% by weight, more preferably from 30% to 53% by weight and especially preferably from 40% to 52% by weight.

In the particular case in which the reactant stream S_{BE2} comprises both water and methanol in addition to M_{B}OH, it is particularly preferable that the proportion by mass of M_{B}OH in methanol and water, based on the total weight of the solution forming S_{BE2}, is especially within a range from 10% to 75% by weight, preferably from 15% to 54% by weight, more preferably from 30% to 53% by weight and particularly preferably from 40% to 52% by weight.

The optional step (a2) of the process according to the invention is performed in a reactive rectification column (or “reaction column”) RR_{B}. Preferred embodiments of the reaction column RR_{B} are described in section 4.1.

According to the invention, the “reaction of a reactant stream S_{BE1} comprising methanol with a reactant stream S_{BE2} comprising M_{B}OH in countercurrent” is achieved more particularly by virtue of the feed point for at least a portion of the reactant stream S_{BE1} comprising methanol in the optional step (a2) being below the feed point of the reactant stream S_{BE2} comprising M_{B}OH in the reaction column RR_{B}.

The reaction column RR_{B} preferably comprises at least 2, in particular 15 to 40, theoretical plates between the feed point of the reactant stream S_{BE1} and the feed point of the reactant stream S_{BE2}.

The reaction column RR_{B} may be operated as a pure stripping column. In that case, the reactant stream S_{BE1} comprising methanol is introduced in vapour form in the lower region of the reaction column RR_{B}.

The optional step (a2) also encompasses the case where a portion of the reactant stream S_{BE1} comprising methanol is added in vapour form below the feed point of the reactant stream S_{BE2} comprising M_{B}OH but nevertheless at the upper end or in the region of the upper end of the reaction column RR_{B}. This makes it possible to reduce the dimensions of the lower region of the reaction column RR_{B}. When a portion of the reactant stream S_{BE1} comprising methanol is added, especially in vapour form, at the upper end or in the region of the upper end of the reaction column RR_{B}, only a fraction of 10% to 70% by weight, preferably of 30% to 50% by weight, (based in each case on the total amount of the methanol used in step (a2)) is fed in at the lower end of the reaction column RR_{B}, and the remaining fraction is added in vapour form in a single stream or divided into a plurality of substreams, preferably 1 to 10 theoretical plates, more preferably 1 to 3 theoretical plates, below the feed point of the reactant stream S_{BE2} comprising M_{B}OH.

In the reaction column RR_{B}, the reactant stream S_{BE1} comprising methanol is then reacted with the reactant stream S_{BE2} comprising M_{B}OH according to the above-described reaction <1> to give M_{B}OCH₃ and H₂O, where these products are present in admixture with the methanol and M_{B}OH reactants since the reaction is an equilibrium reaction. Accordingly, a crude product RP_{B} which contains not only the M_{B}OCH₃ and water products but also methanol and M_{B}OH is obtained in the reaction column RR_{B} in the optional step (a2) of the process according to the invention.

The bottom product stream S_{BP} comprising methanol and M_{B}OCH₃ is then obtained and withdrawn at the lower end of RR_{B}.

The stream of methanol that still contains water, referred to above as “vapour stream S_{BB} comprising water and methanol”, is withdrawn at the upper end of RR_{B}, preferably at the column top of RR_{B}.

This vapour stream S_{BB} comprising water and methanol is directed in step (a3) at least partly into a rectification column RD_{A}, where it is separated by distillation at least partly into a vapour stream S_{OA} comprising methanol, which is withdrawn at the upper end of RD_{A}, and at least one stream S_{UA} comprising water, which is withdrawn at the lower end of RD_{A}. A portion of the methanol obtained in stream S_{OA} in the distillation in step (a3) may be fed to the reaction column RR_{B} as reactant stream S_{BE1}.

In this case, in step (a3) of the process according to the invention, when step (a2) is conducted, at least a portion of the vapour stream S_{BB}, having or having not been mixed with S_{AB} (i.e. in that case separately from S_{AB}), is directed into the rectification column RD_{A}. Preferably, in step (a3) of the process according to the invention, vapour streams S_{BB} and S_{AB} are mixed, and then the mixture is directed into the rectification column RD_{A}.

The amount of methanol encompassed by the reactant stream S_{BE1} is preferably chosen such that it simultaneously serves as solvent for the alkali metal methoxide M_{B}OCH₃ obtained in the bottom product stream S_{BP}. The amount of methanol in the reactant stream S_{BE1} is preferably chosen so as to achieve, in the bottom of the reaction column, the desired concentration of the alkali metal methoxide solution which is withdrawn as bottom product stream S_{BP} comprising methanol and M_{B}OCH₃.

In a preferred embodiment of the process according to the invention, and especially in the cases in which S_{BE2} comprises not only M_{B}OH but also water, the ratio of the total weight (mass; unit: kg) of methanol used as reactant stream S_{BE1} in the optional step (a2) to the total weight (mass; unit: kg) of M_{B}OH used as reactant stream S_{BE2} in the optional step (a2) is 4:1 to 50:1, more preferably 8:1 to 48:1, even more preferably 10:1 to 45:1, yet more preferably 20:1 to 40:1, most preferably 22:1.

The reaction column RR_{B} is operated with or without, preferably with, reflux.

In the embodiment in which a reflux is established in the reaction column RR_{B}, the M_{B}OH used as reactant stream S_{BE2} in the optional step (a2) can also be mixed at least partly with the reflux stream and the resulting mixture can thus be supplied to the optional step (a2).

The optional step (a2) is performed especially at a temperature within a range from 45° C. to 150° C., preferably 47° C. to 120° C., more preferably 60° C. to 110° C., and at a pressure of 0.5 bar abs. to 40 bar abs., preferably within a range from 0.7 bar abs. to 5 bar abs., more preferably within a range from 0.8 bar abs. to 4 bar abs., more preferably within a range from 0.9 bar abs. to 3.5 bar abs., yet more preferably at 1.0 bar abs. to 3 bar abs., most preferably at 1.25 bar abs.

In a preferred embodiment, the reaction column RR_{B} comprises at least one evaporator which is in particular selected from intermediate evaporators V_{ZB} and reboilers V_{SB}. The reaction column RR_{B} more preferably comprises at least one reboiler V_{SB}.

Such an intermediate evaporator V_{ZB} can convert liquid crude product RP_{B} present in the reaction column RR_{B} and comprising M_{B}OCH₃, water, methanol, M_{B}OH to the gaseous state, thus improving the efficiency of the reaction in the optional step (a2) of the process according to the invention.

By virtue of the arrangement of one or more intermediate evaporators V_{ZB} in the upper region of the reaction column RR_{B}, it is possible to reduce the dimensions in the lower region of the reaction column RR_{B}. In the embodiment having at least one, preferably two or more, intermediate evaporators V_{ZB}, it is also possible to introduce substreams of the methanol in liquid form in the upper region of the reaction column RR_{B}.

In the optional step (a2) of the process according to the invention, a bottom product stream S_{BP} comprising methanol and M_{B}OCH₃ is withdrawn at the lower end of the reaction column RR_{B}.

It is preferable that the reaction column RR_{B} comprises at least one reboiler V_{SB} through which the bottom product stream S_{BP} is then directed at least partly and methanol is at least partly removed therefrom, thus affording a bottom product stream S_{BP*} having an elevated proportion by mass of M_{B}OCH₃ compared to S_{BP}.

The proportion by mass of M_{B}OCH₃ in the bottom product stream S_{BP*} is elevated in particular compared to the proportion by mass of M_{B}OCH₃ in the bottom product stream S_{BP} by at least 0.5%, preferably by ≥1%, more preferably by ≥2%, yet more preferably by ≥5%.

It is preferable when S_{BP} or, if at least one reboiler V_{SB} through which the bottom product stream S_{BP} is at least partly directed and methanol is at least partly removed therefrom is used, S_{BP*} has a proportion by mass of M_{B}OCH₃ in methanol within a range from 1% to 50% by weight, preferably 5% to 35% by weight, more preferably 15% to 35% by weight, most preferably 20% to 35% by weight, based in each case on the total mass of S_{BP}/S_{BP*}.

The proportion by mass of residual water in S_{BP}/S_{BP*} is preferably <1% by weight, preferably <0.8% by weight, more preferably <0.5% by weight, based on the total mass of S_{BP}/S_{BP*}.

The proportion by mass of reactant M_{B}OH in S_{BP}/S_{BP*} is preferably <1% by weight, preferably <0.8% by weight, more preferably <0.5% by weight, based on the total mass of S_{BP}/S_{BP*}.

In the embodiments of the present process in which step (a2) is also performed, it is preferable when the bottom product stream S_{AP} is at least partly directed through a reboiler V_{SA} and methanol is at least partly removed from S_{AP} to afford a bottom product stream S_{AP*} having an elevated proportion by mass of M_{A}OCH₃ compared to S_{AP} and/or, preferably and, the bottom product stream S_{BP} is at least partly directed through a reboiler V_{SB} and methanol is at least partly removed from S_{BP} to afford a bottom product stream S_{BP*} having an elevated proportion by mass of M_{B}OCH₃ compared to S_{BP}.

In the embodiments of the present invention in which it is performed, step (a2) of the process according to the invention is performed simultaneously with and spatially separately from step (a1). Spatial separation is ensured by performing steps (a1) and (a2) in the two reaction columns RR_{A} and RR_{B}.

In an advantageous embodiment of the invention, the reaction columns RR_{A} and RR_{B} are accommodated in one column shell, where the column is at least partially subdivided by at least one dividing wall. Such a column having at least one dividing wall will be referred to as “TRD”. Such dividing wall columns are known to the person skilled in the art and are described, for example, in U.S. Pat. No. 2,295,256, EP 0 122 367 A2, EP 0 126 288 A2, WO 2010/097318 A1 and by I. Dejanović, Lj. Matijašević, Ž. Olujić, Chemical Engineering and Processing 2010, 49, 559-580. CN 105218315 A likewise describes dividing wall columns which are used in the rectification of methanol.

In the dividing wall columns suitable for the process according to the invention, the dividing walls preferably extend to the column floor and, in particular, preferably span at least a quarter, more preferably at least a third, yet more preferably at least half, yet more preferably at least two thirds, yet still more preferably at least three quarters, of the column by height. They divide the columns into at least two reaction spaces in which spatially separate reactions may be carried out. The reaction spaces provided by the at least one dividing wall may be of identical or different sizes.

In this embodiment, the bottom product streams S_{AP} and S_{BP} may be withdrawn separately in the respective regions separated by the dividing wall and preferably passed through the reboiler V_{SA}/V_{SB} provided for each reaction space formed by the at least one reaction wall, in which methanol is at least partly removed from S_{AP}/S_{BP} to afford S_{AP*}/S_{BP*}.

In a preferred embodiment of the process according to the invention, accordingly, at least two, more preferably exactly two, of the columns selected from rectification column RD_{A}, reaction column RR_{A} and, if step (a2) is performed, reaction column RR_{B} are accommodated in one column shell, in which case the columns are at least partly separated from one another by a dividing wall extending to the bottom of the column.

In the integrated system composed of reaction column RR_{A} [or, in the embodiments in which step (a2) is performed, reaction column RR_{A} and reaction column RR_{B}] together with rectification column RD_{A} in the process according to the invention, the rectification column RD_{A} is preferably operated at a pressure selected such that the pressure gradient between the columns is low.

The methanol is consumed in the process according to the invention, and especially in a continuous process regime this therefore has to be replaced by fresh methanol.

The fresh methanol is especially fed directly as reactant stream S_{AE1} comprising methanol into the reaction column RR_{A} or, in the embodiments in which step (a2) is performed, into reaction columns RR_{A} and RR_{B}.

In the process according to the invention, it is further preferable to use the methanol-comprising vapour stream S_{OA} partly as reactant stream S_{AE1} in step (a1) and, if step (a2) is conducted, alternatively or additionally as reactant stream S_{BE1} in step (a2). In this preferred embodiment, it is yet more preferable when the fresh methanol is added to the rectification column RD_{A}.

When the fresh methanol is added to the rectification column RD_{A}, it is preferably fed in either in the rectifying section of the rectification column RD_{A} or directly at the top of the rectification column RD_{A}. The optimal feed point depends on the water content of the fresh methanol used, and secondly on the desired residual water content in the vapour stream S_{OA}. The higher the proportion of water in the methanol used, and the higher the purity requirement in the vapour stream S_{OA}, the more advantageous it is to feed it in a few theoretical plates below the top of the rectification column RD_{A}. Up to 20 theoretical plates below the top of the rectification column RD_{A} and in particular 1 to 5 theoretical plates are preferred.

When the fresh methanol is added to the rectification column RD_{A}, it is added at the top of the rectification column RD_{A} at temperatures up to boiling point, preferably at room temperature. A dedicated feed may be provided here for the fresh methanol, or else fresh methanol after the condensation and recycling of a portion of the methanol withdrawn at the top of the rectification column RD_{A} may be mixed therewith and be fed together to the rectification column RD_{A}. In this case it is particularly preferable when the fresh methanol is added to a condensate vessel in which the methanol condensed out of the vapour stream S_{OA} is collected.

As described above, in an advantageous embodiment of the invention, at least two of the columns selected from rectification column RD_{A}, reaction column RR_{A} and, if step (a2) is performed, the reaction column RR_{B} are accommodated in one column shell, in which case the columns are each at least partly separated from one another by a dividing wall extending to the bottom of the column. In the above-described preferred embodiment in which step (a2) is performed, these are accordingly separated from one another by two dividing walls, where the two dividing walls extend to the bottom of the column.

In this preferred embodiment the reaction to afford the crude product RP_{A} according to step (a1) or the crude products RP_{A} and RP_{B} according to steps (a1) and (a2) are in particular performed in one part of the TRD, wherein the reactant stream S_{AE2} and optionally the reactant stream S_{BE2} are added below but at approximately the height of the upper end of the dividing wall and the reactant stream S_{AE1} and optionally the reactant stream S_{BE1} are added in vapour form at the lower end. The methanol/water mixture formed above the feed point of the reactant stream is then distributed above the dividing wall over the entire column region which serves as rectifying section of the rectification column RD_{A}. The second/third lower part of the column which has been separated off by the dividing wall is the stripping section of the rectification column RD_{A}. The energy required for the distillation is then supplied via an evaporator at the lower end of the second portion of the column separated by the dividing wall, and this evaporator may be heated conventionally or heated with a portion of the compressed vapour stream S_{OA2}. When the evaporator is heated conventionally, an intermediate evaporator heated with a portion of the compressed heat transfer medium, e.g. W*₃₁ or W*₄, may additionally be provided.

In the embodiments in which a portion of S_{OA} is used as reactant stream S_{AE1} and/or reactant stream S_{BE1}, S_{OA} is especially compressed with a compressor V_{DAB2}, and in this way the difference in the pressures inside the reaction columns RR_{A} and RR_{B} compared to the pressure in RD_{A} may be taken into account.

Alternatively or additionally, in this preferred embodiment, it is also possible to use, rather than the compressor VD_{AB2} which is downstream of the rectification column RD_{A} and in which S_{OA} is compressed, a compressor VD_{AB1} which is upstream of the rectification column RD_{A} and with which S_{AB}, S_{BB}, or the mixture of S_{AB} and S_{BB} before the respective stream is directed into RD_{A}, is compressed.

### 4.3 Step (a3)

In step (a3) of the process according to the invention, at least a portion of the vapour stream S_{AB}, and, if step (a2) is conducted, at least a portion of the vapour stream S_{BB}, mixed with S_{AB} or separately from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A}.

In the embodiments of the invention in which step (a2) is conducted, it is preferable when, in step (a3), the at least one portion of the vapour stream S_{AB} and the at least one portion of the vapour stream S_{BB} are mixed and then directed into a rectification column RD_{A}. S_{AB} and S_{BB} may alternatively also be directed into the rectification column RD_{A} at two different feed points.

In step (a3) of the process according to the invention, at least a portion of the vapour stream S_{AB}, and, if step (a2) is conducted, at least a portion of the vapour stream S_{BB}, mixed with S_{AB} or separately from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A}.

“At least one vapour stream S_{OA} comprising methanol which is withdrawn at the upper end of RD_{A}” means that the vapour obtained at the upper end of RD_{A} may be withdrawn there as one or more vapour streams. If said stream is withdrawn there in more than one vapour stream, the m vapour streams are referred to as “vapour stream S_{OAI}”, “vapour stream S_{OAII}”, [ . . . ], “vapour stream S_{OAm}”, where “m” indicates the number of vapour streams withdrawn at the upper end of RD_{A} (in Roman numerals).

“At least one stream S_{UA} comprising water which is withdrawn at the lower end of RD_{A}” means that water obtained at the lower end of RD_{A} may be withdrawn there as one or more streams. If said stream is withdrawn there in more than one stream, the n streams are referred to as “stream S_{UAI}”, “stream S_{UAII}”, [ . . . ], “stream S_{UAn}”, where “n” is the number of streams withdrawn at the lower end of RD_{A} (in Roman numerals).

The at least one portion of vapour stream S_{AB} and, when step (a2) is conducted, the at least one portion of vapour stream S_{BB} may be directed into the rectification column RD_{A} via one or more feed points. They are introduced via two or more feed points, for example, in the embodiments in which step (a2) is performed in the process according to the preferred aspect of the invention and, in step (a3), at least a portion of the vapour stream S_{BB} is used separately from S_{AB}. In this embodiment, the at least one portion of vapour stream S_{AB} and the at least one portion of vapour stream S_{BB} are accordingly directed into the rectification column RD_{A} as two separate streams.

In the embodiments of the present invention in which the at least one portion of the vapour stream S_{AB} and, when step (a2) is conducted, the at least one portion of the vapour stream S_{BB} is/are directed into the rectification column RD_{A} as two or more separate streams, it is advantageous when the feed points for the individual streams are at essentially the same height on the rectification column RD_{A}.

In a preferred embodiment of step (a3) of the process according to the invention, the at least one portion of vapour stream S_{AB}, and, when step (a2) is conducted, the at least one portion of vapour stream S_{BB} are separated in the rectification column RD_{A} into a vapour stream S_{OA} comprising methanol, which is withdrawn at the upper end of RD_{A}, and a stream S_{UA} comprising water, which is withdrawn at the lower end of RD_{A}.

Another term for “upper end of a rectification column” is “top”.

Another term for “lower end of a rectification column” is “bottom” or “foot”.

The pressure of the at least one vapour stream S_{OA} is referred to as “p_{OA}” and its temperature as “T_{OA}”. This relates especially to the pressure and temperature of the at least one vapour stream S_{OA} when it is withdrawn from the rectification column RD_{A} in step (a3).

The pressure p_{OA} is especially within a range from 0.5 bar abs. to 8 bar abs., more preferably within a range from 0.6 bar abs. to 7 bar abs., more preferably within a range from 0.7 bar abs. to 6 bar abs., yet more preferably within a range from 1 bar abs. to 5 bar abs., yet more preferably within a range from 1 bar abs. to 4 bar abs., yet more preferably within a range from 1.0 bar abs. to 2.0 bar abs., and is most preferably 1.1 bar abs.

The temperature T_{OA} is especially in the range from 45° C. to 150° C., more preferably in the range from 48° C. to 140° C., more preferably in the range from 50° C. to 130° C., yet more preferably in the range from 60° C. to 120° C., yet more preferably in the range from 60° C. to 110° C., yet more preferably in the range from 65° C. to 80° C., most preferably 67° C.

Any desired rectification column known to those skilled in the art may be used as rectification column RD_{A} in step (a3) of the process. The rectification column RD_{A} preferably contains internals. Suitable internals are, for example, trays, unstructured packings or structured packings. Trays used are typically bubble-cap trays, sieve trays, valve trays, tunnel-cap trays or slotted trays. Unstructured packings are generally beds of random packing elements. Random packing elements used are typically Raschig rings, Pall rings, Berl saddles or Intalox® saddles. Structured packings are sold, for example, under the Sulzer Mellapack® trade name. Apart from the internals mentioned, further suitable internals are known to a person skilled in the art and can likewise be used.

Preferred internals have a low specific pressure drop per theoretical plate. Structured packings and random packing elements have, for example, a significantly lower pressure drop per theoretical plate than trays. This has the advantage that the pressure drop in the rectification column RD_{A} remains as low as possible and the mechanical power of the compressor and the temperature of the methanol/water mixture to be evaporated therefore remain low.

When the rectification column RD_{A} contains structured packings or unstructured packings, these may be divided or in the form of an uninterrupted packing. Typically, however, at least two packings are provided:
1) Preferably,
   when step (a2) is not performed: one packing above the feed point of S_{AB};
   when step (a2) is performed and S_{AB} and S_{BB} are mixed and then directed into RD_{A}: one packing above the feed point of the mixture of S_{AB} and S_{BB};
   when step (a2) is performed and S_{AB} and S_{BB} are directed separately into RD_{A}: one packing above the feed points of S_{AB} and S_{BB}.
2) Preferably, moreover:
   when step (a2) is not performed: one packing below the feed point of S_{AB};
   when step (a2) is performed and S_{AB} and S_{BB} are mixed and then directed into RD_{A}: one packing below the feed point of the mixture of S_{AB} and S_{BB};
   when step (a2) is performed and S_{AB} and S_{BB} are directed separately into RD_{A}: one packing below the feed points of S_{AB} and S_{BB}.

It is also possible to provide one packing above the feed point of S_{AB}/S_{AB} and S_{BB} and multiple trays below the feed point of S_{AB}/S_{AB} and S_{BB}. If an unstructured packing is used, for example a random packing, the random packing elements are typically disposed on a suitable support grid (for example sieve tray or mesh tray).

In the respective embodiment, it is preferable that the feed point of S_{AB}/S_{AB} and S_{BB} is in the lower half of the column RD_{A}, i.e. S_{AB}/S_{AB} and S_{BB} are directed into the lower half of the column RD_{A}.

In step (a3) of the process according to the invention, the at least one vapour stream S_{OA} comprising methanol is then withdrawn at the upper end of the rectification column RD_{A}. The preferred proportion by mass of methanol in this vapour stream S_{OA} is ≥99% by weight, more preferably >99.6% by weight, yet more preferably >99.9% by weight, and the remainder is especially water.

Withdrawn at the lower end of RD_{A} is at least one stream S_{UA} comprising water which may preferably include <1% by weight, more preferably ≤5000 ppmw, yet more preferably ≤2000 ppmw of methanol.

The withdrawal of the at least one vapour stream S_{OA} comprising methanol at the top of the rectification column RD_{A} in the context of the present invention means more particularly that the at least one vapour stream S_{OA} is withdrawn above the internals in the rectification column RD_{A} as a top stream or as a side stream takeoff.

The withdrawal of the at least one stream S_{UA} comprising water at the bottom of the rectification column RD_{A} in the context of the present invention means more particularly that the at least one stream S_{UA} is withdrawn as bottom stream or at the lower tray of the rectification column RD_{A}.

The rectification column RD_{A} is operated with or without, preferably with, reflux.

“With reflux” means that the vapour stream S_{OA} withdrawn at the upper end of the rectification column RD_{A} is not completely discharged but rather partly condensed and returned to the respective rectification column RD_{A}. In the cases where such a reflux is established, the reflux ratio is preferably 0.0001 to 10, more preferably 0.1 to 5, yet more preferably 0.5 to 2, yet more preferably 0.7 to 1, yet more preferably 0.76.

A reflux may be established by mounting a condenser K_{RD} at the top of the rectification column RD_{A}. The vapour stream S_{OA} is partly condensed in the condenser K_{RD} and returned to the rectification column RD_{A}. Generally and in the context of the present invention, a reflux ratio is understood to mean the ratio of the proportion of the mass flow withdrawn from the column (kg/h) that is recycled to the column in liquid form (reflux) to the proportion of this mass flow (kg/h) that is discharged from the respective column in liquid form or gaseous form.

### 4.4 Step (b) of the Process According to the Invention

In step (b) of the process according to the invention, at least one side stream S_{ZA} is withdrawn from RD_{A} and recycled to RD_{A}.

In a preferred embodiment of step (b) of the process according to the invention, a side stream S_{ZA} is withdrawn from RD_{A} and recycled to RD_{A}.

According to the invention, “side stream S_{ZA} from RD_{A}” means that the stream is withdrawn at a withdrawal point E_{ZA} below the top and above the bottom of RD_{A} and in particular additionally recycled to RD_{A} at a feed point Z_{ZA} (this is the point at which the respective side stream S_{ZA} is recycled to the rectification column RD_{A}) below the top and above the bottom of RD_{A}.

This means more particularly that the withdrawal point E_{ZA} and preferably also the feed point Z_{ZA} of the respective side stream S_{ZA} on the rectification column RD_{A} are below the withdrawal points E_{OA} for all vapour streams S_{OA} withdrawn from RD_{A}, preferably at least 1, more preferably at least 5, yet more preferably at least 10, theoretical plates below the withdrawal point E_{OA} for the vapour stream S_{OA} withdrawn from RD_{A} that has the withdrawal point E_{OA} furthest down the rectification column RD_{A}.

This also means more particularly that the withdrawal point E_{ZA}, and preferably also the feed point Z_{ZA} for the respective side stream S_{ZA} on the rectification column RD_{A} are above the withdrawal points E_{UA} for all streams S_{UA} withdrawn from RD_{A}, preferably at least 1, yet more preferably at least 2, yet more preferably at least 4, theoretical plates above the withdrawal point E_{UA} for the stream S_{UA} that has the withdrawal point E_{UA} furthest up the rectification column RD_{A}.

In the cases in which at least one vapour stream S_{OA} is at least partly recycled into the rectification column RD_{A} (which is the case when a reflux is established at the rectification column RD_{A} for example), the feed point Z_{OA} (i.e. the point at which the at least one vapour stream S_{OA} is at least partly recycled into the rectification column RD_{A}) of the at least one vapour stream S_{OA} is especially also above the withdrawal points E_{ZA} and especially also above the feed points Z_{ZA} for all side streams S_{ZA} withdrawn from RD_{A}, preferably at least 1, more preferably at least 5, yet more preferably at least 10, theoretical plates above the highest point of all withdrawal and feed points for all side streams S_{ZA} withdrawn from RD_{A}.

In the cases in which at least one stream S_{UA} is at least partly recycled into the rectification column RD_{A}, moreover, the feed point Z_{UA} (i.e. the point at which the at least one stream S_{UA} is at least partly recycled into the rectification column RD_{A}) of the at least one stream S_{UA} is below the withdrawal points E_{ZA} and especially also below the feed points Z_{ZA} of all side streams S_{ZA} withdrawn from RD_{A}, preferably at least 1, yet more preferably at least 2, yet more preferably at least 4, theoretical plates below the lowest point of all withdrawal and feed points for all side streams S_{ZA} withdrawn from RD_{A}.

The withdrawal point E_{ZA} of the side stream S_{ZA} and the feed point Z_{ZA} of the side stream S_{ZA} on the rectification column RD_{A} may be positioned between the same trays of RD_{A}. However, they may also be at different heights.

In a preferred embodiment of the process according to the invention, the withdrawal point E_{ZA} and preferably also the feed point Z_{ZA} of the at least one side stream S_{ZA} on the rectification column RD_{A} are below the feed point Z_{SAB}, and above the bottom of RD_{A}. It is yet more preferable when the withdrawal point E_{ZA} and preferably also the feed point Z_{ZA} of the at least one side stream S_{ZA} on the rectification column RD_{A} are also below the rectifying section of RD_{A}.

The feed point Z_{SAB} refers to the lowermost feed point of all feed points of S_{AB} into RD_{A}, of all feed points of S_{BB} into RD_{A}, and of all feed points of the mixture of S_{AB} and S_{BB} into RD_{A}.

In a particularly preferred embodiment of the process according to the invention, the withdrawal point E_{ZA} and more preferably also the feed point Z_{ZA} of the at least one side stream S_{ZA} on the rectification column RD_{A} are in the upper ⅘, preferably upper ¾, preferably upper 7/10, more preferably upper ⅔, more preferably upper ½, of the region on the rectification column RD_{A} below the feed point Z_{SAB} and above the uppermost of all withdrawal and feed points for all streams S_{UA} withdrawn from RD_{A}.

It is yet more preferable when the withdrawal point E_{ZA} and preferably also the feed point Z_{ZA} of the at least one side stream S_{ZA} on the rectification column RD_{A} are then also below the rectifying section of RD_{A}.

In a further particularly preferred embodiment of the process according to the invention, rectification column RD_{A} contains a rectifying section, and the withdrawal point E_{ZA} and more preferably also the feed point Z_{ZA} of the at least one side stream S_{ZA} on the rectification column RD_{A} are in the upper ⅘, preferably upper ¾, preferably upper 7/10, more preferably upper ⅔, more preferably upper ½, of the region on the rectification column RD_{A} below the rectifying section and above the uppermost of all withdrawal and feed points for all streams S_{UA} withdrawn from RD_{A}.

### 4.5 Step (c) of the Process According to the Invention

In step (c) of the process according to the invention, energy, preferably heat, is transferred from at least a portion of S_{OA} to a liquid or gaseous, preferably liquid, heat transfer medium W*₁, which affords a gaseous heat transfer medium W*₂.

The heat transfer medium W*₁ utilized may be any working medium familiar to the person skilled in the art. The heat transfer medium W*₁ is especially selected from the group consisting of water, optionally fluorinated alkanes, ammonia, alcohols. Preferably, W*₁ is selected from n-hexane, n-pentane, n-butane, n-propane, methanol, ethanol, propanol. Most preferably, W*₁=n-butane.

Step (c) of the process according to the invention transfers energy, preferably heat, from at least a portion of S_{OA} to the liquid or gaseous, preferably liquid, heat transfer medium W*₁, and hence the gaseous heat transfer medium W*₂ is obtained.

This means that, when the heat transfer medium W*₁ is used in liquid form in step (c), energy, preferably heat, is supplied thereto in step (c), such that the liquid heat transfer medium W*₁ at least partly evaporates, and hence a gaseous heat transfer medium W*₂ is obtained.

What is meant more particularly by “liquid heat transfer medium W*₁” is that ≥10% by weight of the heat transfer medium W*₁ used in step (c) is in the liquid state of matter, based on the total weight of the heat transfer medium W*₁ used in step (c). It preferably means that ≥25% by weight, more preferably ≥50% by weight, more preferably ≥55% by weight, more preferably ≥75% by weight, more preferably >90% by weight, more preferably >99% by weight, of the heat transfer medium W*₁ used in step (c) is in the liquid state of matter, based on the total weight of the heat transfer medium W*₁ used in step (c).

If a liquid heat transfer medium W*₁ is used in step (c) of the process according to the invention, in one preferred embodiment a sufficient amount of energy, preferably heat, is transferred in step (c) of the process according to the invention from at least a portion of S_{OA} to the liquid heat transfer medium W*₁ that, during step (c), ≥10% by weight, preferably ≥20% by weight, preferably ≥30% by weight, preferably ≥40% by weight, preferably ≥50% by weight, preferably ≥60% by weight, preferably ≥70% by weight, preferably ≥80% by weight, preferably ≥90% by weight, preferably ≥99% by weight, of the heat transfer medium W*₁ used in the liquid state of matter is converted to the gaseous state, more preferably that, during step (c), the liquid heat transfer medium W*₁ is converted completely to the gaseous state.

Alternatively, the heat transfer medium W*₁ may be used in gaseous form in step (c). In that case, since it is supplied with energy, preferably heat, in step (c), a gaseous heat transfer medium W*₂ is obtained after step (c).

What is meant more particularly by “gaseous heat transfer medium W*₁” is that the heat transfer medium W*₁ used in step (c) is entirely in the gaseous state of matter.

According to the invention, “transfer of energy” especially means “heating”, i.e. transfer of energy in the form of heat.

“Transfer of energy, preferably heat, from at least a portion of S_{OA} to a liquid or gaseous, preferably liquid, heat transfer medium W*₁” also encompasses the embodiments of step (c) in which S_{OA} is first divided, for example into a portion S_{OA1} which, optionally after compression of this portion S_{OA1}, is used as methanol stream S_{AE1} and/or S_{BE1}, and a portion S_{OA2} which is recycled to the column RD_{A} as return stream, in which case energy, especially heat, is transferred solely from S_{OA2} to W*₁.

W*₂ has an elevated energy content compared to W*₁. By contrast, there is a drop in the energy content of S_{OA} during step (c).

The transfer of energy, especially heat, from at least a portion of S_{OA} to the liquid or gaseous, preferably liquid, heat transfer medium W*₁ is preferably direct or indirect, more preferably direct. Another word for “transfer of heat” is “heating”.

What is meant more particularly by “directly” is that S_{OA} is contacted with W*₁ without mixing of S_{OA} and W*₁, such that energy, especially heat, is transferred from S_{OA} to W*₁.

This can be performed with the aid of methods known to the person skilled in the art.

In particular, S_{OA} and W*₁ can be directed through a heat transferrer in which energy, preferably heat, is transferred from S_{OA} to W*₁.

Heat transferrers used (another term for “heat transferrer”=“heat exchanger”) may be the heat transferrers that are familiar to the person skilled in the art, especially evaporators, especially kettle evaporators, as described above (in point 4.1). Preference is given here to using a kettle evaporator in which W*₁ is expanded and, thereafter or at the same time, energy is absorbed from S_{OA}.

As described above, in a preferred embodiment of the present invention, a return stream is established in the rectification column RD_{A}, in which case the vapour stream S_{OA} is partly condensed in a condenser K_{RD} mounted at the top of the rectification column RD_{A} in particular, and fed back to the rectification column RD_{A}.

In this preferred embodiment, the direct transfer of energy, preferably heat, from S_{OA} to W*₁ is undertaken in the condenser K_{RD} in particular. This has the advantage that the condenser K_{RD} can be used simultaneously as heat transferrer, and there is no need to install any additional evaporator/condenser.

“Indirectly” means more particularly that S_{OA} is contacted with a heat transfer medium W₁ other than W*₁, preferably via at least one heat transferrer WTₓ, where the heat transfer medium W₁* is not W*₁, and [symbol] is thus distinct from W*₁, and so energy, preferably heat, is transferred from S_{OA} to W₁ without mixing of the two streams, and the heat is then transferred from W₁ to [symbol] in that the heat transfer medium [symbol] comes into contact with the heat transfer medium W*₁, with or without mixing of W*₁ and [symbol] , but preferably without. If [symbol] and W*₁ do not mix, the energy, preferably heat, is transferred especially in a further heat transferrer WT_{Y}.

In a further embodiment of the process according to the invention, in the case of indirect energy transfer from S_{OA} to W*₁, in particular heating of W*₁ by S_{OA}, it is also possible for energy, preferably heat, first to be transferred from S_{OA} to [symbol] , preferably by contacting via at least one heat exchanger WTₓ, and then transferred from [symbol] to a further heat transfer medium [symbol] distinct from W*₁, preferably by contacting via at least one heat exchanger WT_{Y}. The last step then transfers the heat from [symbol] to W*₁, with or without mixing of W*₁ and [symbol] , but preferably without. If [symbol] and W*₁ do not mix, the energy, preferably heat, is transferred especially in a further heat transferrer WT_{Z}.

It will be apparent that still further heat transfer media [symbol] , [symbol] , [symbol] etc. may accordingly be utilized in further embodiments of the present invention.

Utilizable heat transfer media [symbol] and additionally further utilized heat transfer media [symbol] , [symbol] , [symbol] , [symbol] include any heat transfer media known to those skilled in the art, preferably selected from the group consisting of air; water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium [symbol] used is water or air, most preferably water.

As described above, in a preferred embodiment of the present invention, a return stream is established in the rectification column RD_{A}, in which case the vapour stream S_{OA} is partly condensed in a condenser K_{RD} mounted at the top of the rectification column RD_{A} in particular, and fed back to the rectification column RD_{A}.

In this preferred embodiment, the direct or indirect transfer of energy, preferably heat, from S_{OA} to W*₁ or S_{OA} to [symbol] is undertaken in the condenser K_{RD} in particular. This has the advantage that the condenser K_{RD} can be used simultaneously as heat transferrer, and there is no need to install any additional evaporator/condenser.

After step (c) of the process according to the invention, a gaseous heat transfer medium W*₂ is obtained.

The pressure of W*₂ is referred to as “p_{W*2}” and its temperature as “T_{W*2}”.

The pressure of W*₁ is referred to as “p_{W*1}” and its temperature as “T_{W*1}”.

In a preferred embodiment of the present invention, W*₁ is at a temperature T_{W*1} in the range from 50° C. to 170° C., more preferably 90° C., and, especially when W*₁ is gaseous, at a pressure p_{W*1} of 1 bar to 35 bar, more preferably 1.5 bar to 20 bar.

In a preferred embodiment of the present invention, W*₂ is at a temperature T_{W*2} in the range from 25° C. to 150° C., more preferably 70° C., and at a pressure p_{W*2} of 1 bar to 35 bar, more preferably 5 bar to 8 bar, even more preferably 6.4 to 6.7 bar.

It will be apparent that the heat transfer medium W*₂ is the same as the heat transfer medium W*₁, and W*₂ and W*₁ differ only in their respective pressures p_{W*2} and p_{W*1} and/or their temperature T_{W*2} and T_{W*1}, and, as the case may be, when W*₁ has been used in liquid form, by their state of matter.

### 4.6 Step (d) of the Process According to the Invention

In step (d) of the process according to the invention, at least a portion of the gaseous heat transfer medium W*₂ is compressed. This affords a compressed gaseous heat transfer medium W*₃ relative to W*₂.

The pressure of W*₂ is referred to as “p_{W*2}” and its temperature as “T_{W*2}”.

The pressure of W*₃ is referred to as “p_{W*3}” and its temperature as “T_{W*3}”.

The pressure p_{W*3} is higher than p_{W*2}. The exact value of p_{W*3} can be adjusted by the person skilled in the art according to the requirements in step (d) provided that the condition p_{W*3}>p_{W*2} is met. The quotient of p_{W*3}/p_{W*2} (pressures each in bar abs.) is preferably in the range from 1.1 to 10, more preferably 1.2 to 8, more preferably 1.25 to 7, more preferably 1.3 to 6, yet more preferably 1.5 to 2, yet more preferably 1.6 to 1.8, most preferably 1.7.

The temperature T_{W*3} is especially higher than the temperature T_{W*2}, and the quotient of T_{W*3}/T_{W*2} (temperature in ° C. in each case) is preferably in the range from 1.03 to 10, more preferably 1.04 to 9, more preferably 1.05 to 8, more preferably 1.06 to 7, more preferably 1.07 to 6, most preferably 1.08 to 5.

The preferred values of p_{W*3} and T_{W*3} are also correspondingly applicable to the preferred pressure and the preferred temperature of W*₃₁ and W*₃₂.

The at least one portion of the gaseous heat transfer medium W*₂ can be compressed in step (d) in any desired manner known to the person skilled in the art. For example, the compression can be performed mechanically and as a single-stage or multistage compression, preferably a multistage compression. In a multistage compression, it is possible to use two or more compressors of the same type or compressors of different types. A multistage compression can be effected with one or more compressors. The use of single-stage compression or multistage compression depends on the compression ratio and thus on the pressure to which the gaseous heat transfer medium W*₂ is to be compressed.

The compressor used in the process according to the invention, especially for compression of the gaseous heat transfer medium W*₂ to W*₃ or W*₃₂ to W*₄, is any desired compressor, preferably mechanical compressor, which is known to the person skilled in the art and with which gas streams can be compressed. Suitable compressors are, for example, single-stage or multistage turbines, piston compressors, screw compressors, centrifugal compressors or axial compressors.

In a multistage compression, compressors suitable for the respective pressure stages to be overcome are used.

### 4.7 Step (e) of the Process According to the Invention

In step (e) of the process according to the invention, energy, especially heat, is transferred from a first portion W*₃₁ of the gaseous heat transfer medium W*₃ to S_{ZA} before S_{ZA} is recycled into RD_{A}.

The gaseous heat transfer medium W*₃ is first divided into at least two portions W*₃₁ and W*₃₂, especially in step (e). The ratio of the mass flows (in kg/h) of W*₃₁ to W*₃₂ is preferably in the range from 1:99 to 99:1, more preferably in the range from 1:50 to 50:1, yet more preferably in the range from 1:20 to 30:1, yet more preferably in the range from 5:20 to 15:1, more preferably 2:1 to 5:1, yet more preferably 4:1 to 4.5:1, most preferably 4.4:1.

In step (e) of the process according to the invention, energy is transferred from the first portion W*₃₁ to S_{ZA}. Step (e) lowers the energy of W*₃₁, such that W*₃₁ in particular is at least partly condensed.

According to the invention, “transfer of energy” especially means “heating”, i.e. transfer of energy in the form of heat.

“Transfer of energy from a first portion W*₃₁ of the compressed vapour stream W*₃ to S_{ZA}” also encompasses the cases in which a portion of W*₃₁ is separated off and energy is transferred to S_{ZA} solely from that portion. This is the case for example in embodiments of the invention in which energy is additionally transferred from W*₃₁ to the crude product RP_{A} and, if step (a2) is performed, alternatively or additionally to the crude product RP_{B} (described in section 4.1).

The transfer of energy from W*₃₁ to S_{ZA}, preferably the heating of S_{ZA} by W*₃₁, is preferably direct or indirect.

“Direct” means that W*₃₁ is contacted with S_{ZA} without mixing of the two streams, and so energy, especially heat, is transferred from W*₃₁ to S_{ZA}.

This can be implemented in that W*₃₁ and S_{ZA} are directed through an intermediate evaporator V_{ZRD} in the rectification column RD_{A}, and W*₃₁ heats S_{ZA}.

Heat transferrers used (another term for “heat transferrer”=“heat exchanger”), especially the heat transferrers WTₓ, WT_{Y}, WT_{Z} mentioned hereinafter, may be the heat transferrers, especially evaporators, that are familiar to the person skilled in the art. In step (e) of the process according to the invention, energy, more preferably heat, is in particular transferred from W*₃₁ to S_{ZA} in an intermediate evaporator V_{ZRD}.

“Indirect” means more particularly that W*₃₁ is contacted with a heat transfer medium [symbol] , preferably by means of at least one heat transferrer WT_{X}, where the heat transfer medium [symbol] is not S_{ZA}, i.e. [symbol] is different from S_{ZA}, such that energy, preferably heat, is transferred from W*₃₁ to [symbol] without mixing of the two streams, and the heat is then transferred from [symbol] to S_{ZA} in that [symbol] is contacted with stream S_{ZA}, with or without mixing of S_{ZA} and [symbol] , but preferably without mixing. If [symbol] and S_{ZA} do not mix, energy, preferably heat, is transferred especially in a further heat transferrer WT_{Y}.

In a further embodiment of the process according to the invention, in the case of indirect energy transfer from W*₃₁ to S_{ZA}, especially heating of S_{ZA} by W*₃₁, it is also possible for energy, preferably heat, first to be transferred from W*₃₁ to [symbol] , preferably by contacting via at least one heat exchanger WTₓ, and then transferred from [symbol] to a further heat transfer medium [symbol] distinct from S_{ZA}, preferably by contacting via at least one heat exchanger WT_{Y}. The last step then transfers the heat from [symbol] to S_{ZA}, with or without mixing of S_{ZA} and [symbol] , but preferably without mixing. If [symbol] and S_{ZA} do not mix, the energy, preferably heat, is transferred in a further heat transferrer WT_{z} in particular.

It will be apparent that still further heat transfer media [symbol] , [symbol] , [symbol] etc. may accordingly be utilized in further embodiments of the present invention.

Utilizable heat transfer media [symbol] and additionally further utilized heat transfer media [symbol] , [symbol] , [symbol] , [symbol] include any heat transfer media known to those skilled in the art, preferably selected from the group consisting of air, water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium [symbol] used is water or air, yet more preferably water.

Salt-water solutions that may be used are also described for example in DE 10 2005 028 451 A1 and WO 2006/134015 A1.

In a preferred embodiment, energy transfer from W*₃₁ continues, especially after the transfer of energy to S_{ZA}.

In a preferred embodiment of the process according to the invention, energy, preferably heat, is transferred from W*₃₁, after W*₃₁ has transferred energy to S_{ZA} as per step (e), to S_{OA} or a portion of S_{OA}, especially to the portion of S_{OA}, S_{OA1} which is sent to a compression, and this may be a precompression of the portion of S_{OA}. This makes it possible to use a portion of the residual energy or residual heat still stored by W*₃₁ in the process, in this case for heating of S_{OA} or of a portion of S_{OA}, S_{OA1}.

“Precompression” refers in particular to the first compression stage in the case of a multistage compression.

Other, preferred additional sinks for the energy, preferably heat, in W*₃₁ are described further down (see paragraph 4.10).

Step (e) of the process according to the invention reflects one aspect of the unexpected effect of the present invention. This is that the surplus energy obtained in the compression of the gaseous heat transfer medium W*₂ to give the compressed gaseous heat transfer medium W*₃ does not dissipate without being utilized, but instead is used in the rectification. This is effected in such a way that W*₂ is first compressed to give W*₃, which enables adjustment to the value optimal for energy transfer from W*₃₁ to S_{ZA}, and then a portion W*₃₂ that is distinct from W*₃₁ may be compressed further to give W*₄. The heat of condensation obtained in the further compression of W*₃₂ further to give W*₄ is introduced into the column in the reboiler. The required additional compressor power is less than the heating steam power saved thereby. The process according to the invention requires less energy than that of the prior art, as shown in examples 1 and 2. Compression to W*₄ allows the pressure and temperature of W*₄ to be adjusted such that optimal energy transfer from W*₄ to S_{UA1} or S_{UA} is possible.

### 4.8 Step (f) of the Process According to the Invention

In step (f) of the process of the invention, a portion of the gaseous heat transfer medium W*₃ other than W*₃₁, W*₃₂, is compressed further to obtain a vapour stream W*₄ that has been compressed relative to W*₃₁.

It will be apparent that W*₄, after performance of step (f), is also compressed relative to W*₃₂ and W*₃.

The pressure of the vapour stream W*₄ is referred to as “p_{W*4}” and its temperature as “T_{W*4}”.

The pressure p_{W*4} is higher than p_{W*3}, and the quotient of p_{W*4}/p_{W*3} (pressures each in bar abs.) is preferably in the range from 1.1 to 10, more preferably 1.2 to 8, more preferably 1.25 to 7, more preferably 1.3 to 6, more preferably 1.4 to 5, more preferably 1.5 to 2, more preferably 1.5 to 1.8, most preferably 1.61.

The temperature T_{W*4} is especially higher than the temperature T_{W*3}, and the quotient of T_{W*4}/T_{W*3} (temperature in ° C. in each case) is preferably in the range from 1.03 to 10, more preferably 1.04 to 9, more preferably 1.05 to 8, more preferably 1.06 to 7, more preferably 1.07 to 6, most preferably 1.08 to 5.

The compressing of W*₃₂ in step (f) may be performed by processes familiar to those skilled in the art. For instance, the compression may be performed mechanically and as a single-stage or multistage compression, preferably a multistage compression. In a multistage compression, it is possible to use two or more compressors of the same type or compressors of different types. The use of single-stage compression or multi-stage compression depends on the pressure to which the vapour W*₃₂ is to be compressed. The embodiments of the compression and also the preferred compressor types that have been described in the context of step (d) for W*₂ may also be used for the compression of W*₃₂ to give W*₄, but compression in one stage is sufficient in step (f) in particular, i.e. using a compressor VD_{X}.

### 4.9 Step (g) of the Process According to the Invention

In step (g) of the process according to the invention, energy is transferred from at least a portion of W*₄ to at least a portion S_{UA1} of the at least one stream S_{UA} before S_{UA1} is recycled into RD_{A}.

Preferably, in step (g), energy is transferred from at least a portion of W*₄ to a portion S_{UA1} of the at least one stream S_{UA} before S_{UA1} is recycled into RD_{A}.

Step (g) lowers the energy of W*₄, such that W*₄ in particular is at least partly condensed.

Step (g) of the process according to the invention is preferably performed according to the following embodiments (g1), (g2), (g3):
(g1) energy is transferred from at least a portion of W*₄ to a portion S_{UA1} of the at least one stream S_{UA}, and S_{UA1} is then recycled into RD_{A};
(g2) energy is transferred from at least a portion of W*₄ to a portion S_{UA1*} of the at least one stream S_{UA}, and then a portion S_{UA1} of S_{UA1*} is recycled into RD_{A};
(g3) energy is transferred from at least a portion of W*₄ to the whole of stream S_{UA} and then the whole of stream S_{UA} or only a portion S_{UA1} of stream S_{UA}, preferably only a portion S_{UA1} of stream S_{UA}, is recycled into RD_{A}.

The transfer of energy from at least a portion of W*₄ to the at least a portion S_{UA1} of the at least one stream S_{UA}, preferably the heating of the at least a portion S_{UA1} of the at least one stream S_{UA} by at least a portion of W*₄, is preferably direct or indirect.

“Direct” means that at least a portion of W*₄ is contacted with the at least a portion S_{UA1} of the at least one stream S_{UA} without mixing of the two streams, and so energy, especially heat, is transferred from at least a portion W*₄ to the at least a portion S_{UA1} of the at least one stream S_{UA}.

This may be performed in that the at least a portion of W*₄ and the at least a portion S_{UA1} of the at least one stream S_{UA} are directed through a reboiler V_{SRD} in the rectification column RD_{A}, and W*₄ heats the at least a portion S_{UA1} of the at least one stream S_{UA}.

Heat transferrers used, in particular the heat transferrers WT_{X}, WT_{Y}, WT_{Z} mentioned hereinafter, may be the heat exchangers familiar to the person skilled in the art, in particular evaporators. In step (g) of the process according to the invention, the energy, preferably heat, is in particular transferred from at least a portion of W*₄ to the at least a portion S_{UA1} of the at least one stream S_{UA} in a reboiler V_{SRD}.

“Indirect” means more particularly that the at least a portion of W*₄ is contacted with at least one heat transfer medium W^{♥}₁, preferably by means of at least one heat exchanger WT_{X}, where the heat transfer medium is not the at least a portion S_{UA1} of the at least one stream S_{UA}, i.e. W^{♥}₁ is distinct therefrom, such that energy, preferably heat, is transferred from the at least a portion of W*₄ to the at least one heat transfer medium W^{♥}₁ without mixing of the two streams, and the heat is then transferred from W^{♥}₁ to the at least a portion S_{UA1} of the at least one stream S_{UA} in that W^{♥}₁ makes contact with stream S_{UA1} with or without mixing of the at least a portion S_{UA1} of the at least one stream S_{UA} and W^{♥}₁, but preferably without mixing.

In a further embodiment of the process according to the invention, in the case of indirect energy transfer from the at least a portion of W*₄ to the at least a portion S_{UA1} of the at least one stream S_{UA}, in particular heating of the at least a portion S_{UA1} of the at least one stream S_{UA} by the at least a portion of W*₄, it is also possible for energy, preferably heat, first to be transferred from W*₄ to W^{♥}₁, preferably by contacting via at least one heat exchanger WT_{X}, and then transferred from [symbol] to a further heat transfer medium W^{♥}₂ distinct from the at least a portion S_{UA1} of the at least one stream S_{UA}, preferably by contacting via at least one heat exchanger WT_{Y}. The last step then transfers the heat from W^{♥}₂ to the at least a portion S_{UA1} of the at least one stream S_{UA}, with or without mixing of the at least a portion S_{UA1} of the at least one stream S_{UA} and W₂, but preferably without.

It will be apparent that still further heat transfer media W^{♥}₃, W^{♥}₄, W^{♥}₅ etc. may accordingly be utilized in further embodiments of the present invention.

Utilizable heat transfer media W^{♥}₁ and additionally further utilized heat transfer media W^{♥}₂, W^{♥}₃, W^{♥}₄, W^{♥}₅ include any heat transfer media known to those skilled in the art, preferably selected from the group consisting of air; water; alcohol-water solutions; salt-water solutions, also including ionic liquids, for example LiBr solutions, dialkylimidazolium salts such as, in particular, dialkylimidazolium dialkylphosphates; mineral oils, for example diesel oils; thermal oils, for example silicone oils; biological oils, for example limonene; aromatic hydrocarbons, for example dibenzyltoluene. Most preferably, the heat transfer medium W^{♥}₁ used is water or air, yet more preferably water.

Salt-water solutions that may be used are also described for example in DE 10 2005 028 451 A1 and WO 2006/134015 A1.

In a preferred embodiment, after step (g), transfer of energy from at least a portion of W*₄ is continued, especially after the transfer of the energy to the at least a portion S_{UA1} of S_{UA}.

In a preferred embodiment of the process according to the invention, energy, more preferably heat, is transferred from at least a portion of W*₄, after W*₄ has transferred energy to the at least a portion S_{UA1} of S_{UA} as per step (g), to S_{OA} or a portion of S_{OA}, especially to the portion of S_{OA}, S_{OA1} which is sent to a compression, and this may be a precompression of the portion of S_{OA}. This makes it possible to use a portion of the residual energy or residual heat still stored by the at least a portion W*₄ in the process, in this case for heating of S_{OA} or of a portion of S_{OA}, S_{OA1}.

After step (g), at least a portion of W*₄ can then be combined again with the heat transfer medium W*₃, W*₃₁, W*₃₂ obtained after performance of step (d) or (e) and sent as liquid or gaseous heat transfer medium W*₁ to a new cycle of the process in step (c). Optionally, W*₄ is expanded before being combined with one of streams W*₃, W*₃₁, W*₃₂.

In a preferred embodiment of the invention, a portion of the heat transfer medium W*₄ obtained after step (g) is also expanded to a lower pressure by means of a valve before being fed to a new cycle as W*₁. This can lower the pressure from W*₄ to the preferred range of W*₁.

Alternatively or additionally to a valve, energy, especially heat, may be transferred from at least a portion of the heat transfer medium W*₄ obtained after step (g) to W*₂ before W*₂ is compressed in step (d).

Alternatively or additionally, a portion of the heat transfer medium W*₄ obtained after step (g) may also be expanded by a valve or into a condensate vessel, and the portion thus expanded may then be combined with W*₃, especially one of the portions W*₃₁ or W*₃₂ of the gaseous heat transfer medium W*₃.

Other, preferred additional sinks for the energy, preferably heat, in the at least a portion of W*₄ are described further down (see paragraph 4.10).

In a further preferred embodiment, energy is transferred from the bottom product stream S_{AP} and, if step (a2) is conducted, alternatively or additionally from the bottom product stream S_{BP} to S_{OA} or a portion of S_{OA}, especially to the portion of S_{OA}, S_{OA}1 which is sent to a compression, and this may be a precompression of the portion of S_{OA}.

In a further preferred embodiment, energy is transferred from the bottom product stream S_{AP} and, if step (a2) is conducted, alternatively or additionally from the bottom product stream S_{BP} to at least a portion of W*₁ before W*₁ is used in step (c).

### 4.10 Preferred Aspect: Process for Transalcoholization of an Alkali Metal Alkoxide

In an advantageous embodiment of the present invention, the energy encompassed in at least one of the streams W*₃, W*₃₁, W*₃₂, W*₄ is used for operation of other industrial processes. This is advantageous especially in sites hosting integrated systems (chemistry parks, technology parks) where there is always a need for heating. This energy may be advantageously utilized especially in the case of integrated systems comprising two or more plants for alkali metal alkoxide production. Such integrated systems typically also comprise processes for transalcoholization as described in DE 27 26 491 A1. U.S. Pat. No. 3,418,383 A, WO 2021/122702 A1 describe processes for transalcoholization from methoxides to propoxides.

In a preferred aspect of the present invention, in the process according to the present invention, in a reactive rectification column RR_{C} a reactant stream S_{CE1} comprising M_{c}OR′, with or without R′OH, is reacted in countercurrent with a reactant stream S_{CE2} comprising R″OH to afford a crude product RP_{C} comprising M_{c}OR″ and R′OH,
wherein a bottom product stream S_{cp} comprising M_{c}OR″ is withdrawn at the lower end of RR_{c} and a vapour stream S_{CB} comprising R′OH is withdrawn at the upper end of RR_{c},
and wherein R′ and R″ are two different C₁ to C₆ hydrocarbon radicals, and M_{c} is a metal selected from lithium, sodium, potassium, preferably sodium, potassium, more preferably sodium,
and wherein energy is transferred from at least a portion of a stream selected from W*₃, W*₄ to the crude product RP_{C}.

The process according to the invention in the preferred aspect of the invention is a process for transalcoholization of a given alkali metal alkoxide M_{c}OR′ to afford another alkali metal alkoxide M_{c}OR″ as described for example in DE 27 26 491 A1 or WO 2021/122702 A1.

R′ and R″ here are two different C₁ to C₆ hydrocarbon radicals, preferably two different C₁ to C₄ hydrocarbon radicals.

Yet more preferably, R′ is methyl and R″ is a C₂ to C₄ hydrocarbon radical.

Yet more preferably, R′ is methyl and R″ is selected from ethyl, n-propyl, iso-propyl, sec-butyl, 2-methyl-2-butyl, tert-butyl, 2-methyl-2-pentyl, 3-methyl-3-pentyl, 3-ethyl-3-pentyl, 2-methyl-2-hexyl, 3-methyl-3-hexyl, especially from ethyl, iso-propyl, 2-methyl-2-butyl, 3-methyl-3-pentyl, 3-ethyl-3-pentyl.

Even more preferably, R′=methyl and R″=ethyl.

The process according to the preferred aspect of the invention (also referred to hereinafter as “transalcoholization”) is performed in a reactive rectification column RR_{C}. Suitable reactive rectification columns include columns described for RR_{A} at point 4.1 in the context of step (a1).

The reaction column RR_{C} is operated with or without, preferably with, reflux. If a reflux is established, the vapour S_{CB} in particular is directed partly or completely through a condenser K_{RRC}, and the condensed vapour may then be returned to the reaction column RR_{C} or, when R′=methyl, used as reactant stream S_{AE1} or S_{BE1}. When R′=methyl, it may also be used as fresh alcohol stream in RD_{A}.

In the transalcoholization, a bottom product stream S_{CP} comprising M_{C}OR″ is withdrawn at the lower end of RR_{C}. A vapour stream S_{CB} comprising R′OH is withdrawn at the upper end of RR_{C}.

In a preferred embodiment, when R′=methyl, the reactant stream S_{CE1} used, comprising M_{c}OR′ with or without R′OH, is at least a portion of S_{AP}, and, when step (a2) is conducted, alternatively (when M_{A} and M_{B} are different alkali metals or when M_{A} and M_{B} are the same alkali metal, especially when M_{A} and M_{B} are different alkali metals) or additionally (especially when M_{A} and M_{B} are the same alkali metal), at least a portion of S_{BP} is used. More preferably, in that case, R″=ethyl. What accordingly takes place is a transalcoholization of alkali metal methoxide to the corresponding alkali metal ethoxide.

When S_{BP} and S_{AP} comprise the same alkali metal methoxide, these two streams may also be used separately or in mixed form as S_{CE1}, i.e. in particular first mixed and then fed to the column RR_{C} as reactant stream S_{CE1} or fed to the column RR_{c} separately as two reactant streams S_{CE1}.

The reactant stream S_{CE2} comprises R″OH. In a preferred embodiment, the proportion by mass of R″OH in S_{CE2} is ≥85% by weight, yet more preferably ≥90% by weight, where S_{CE2} otherwise includes M_{C}OR″ in particular or another denaturing agent. The alcohol R″OH used as reactant stream S_{CE2} may also be commercially available alcohol having a proportion by mass of alcohol of more than 99.8% by weight and a proportion by mass of water of up to 0.2% by weight.

According to the invention, “reaction of a reactant stream S_{CE1} comprising M_{c}OR′, with or without R′OH, with a reactant stream S_{CE2} comprising R″OH in countercurrent” is achieved more particularly by virtue of the feed point for at least a portion of the reactant stream S_{CE1} comprising M_{c}OR′ being above the feed point of the reactant stream S_{CE2} comprising R″OH in the reaction column RR_{C}.

The reaction column RR_{C} is operated with or without, preferably with, reflux.

In a preferred embodiment, the reaction column RR_{c} comprises at least one evaporator which is in particular selected from intermediate evaporators V_{ZC} and reboilers V_{SC}. The reaction column RR_{C} more preferably comprises at least one reboiler V_{SC}.

In the case of the reaction column RR_{C}, intermediate evaporation comprises withdrawal (“takeoff”) of at least one side stream S_{ZC} from RR_{C} and feeding thereof to the at least one intermediate evaporator V_{ZC}.

In the case of the reaction column RR_{C}, bottoms evaporation comprises withdrawal (“takeoff”) of at least one stream, for example S_{CP} from RR_{C}, and feeding of at least a portion, in the case of S_{CP} preferably a portion, to the at least one reboiler V_{SC}.

Suitable evaporators that can be used as intermediate evaporators and reboilers are described in section 4.1.

In the transalcoholization, energy, preferably heat, is transferred from at least a portion of a stream selected from W*₃, W*₄ to the crude product RP_{C}. This is preferably effected by transfer of energy from at least a portion of a stream selected from W*₃, W*₄ to S_{CE1} or S_{CE2} before they are directed into RR_{C}, followed by transfer of energy from S_{CE1}/S_{CE2} to the crude product RP_{C} present in RR_{C}, with which they are mixed.

Accordingly, energy, preferably heat, is transferred from at least a portion of a heat transfer medium selected from W*₃, W*₄, especially from at least one heat transfer medium selected from W*₃₁, W*₃₂, W*₄, preferably from at least one heat transfer medium selected from W*₃₂, W*₄, to the crude product RP_{C}.

“Transfer of energy, preferably heat, from at least a portion of W*₃ to the crude product RP_{C}” also encompasses the transfer of energy, preferably heat, from at least one heat transfer medium selected from W*₃₁, W*₃₂, W*₃, before separation thereof into W*₃₁, W*₃₂, to the crude product RP_{C}.

In addition, it is also possible to direct crude product RP_{C} through an intermediate evaporator V_{ZC} or a reboiler V_{SC} and, in V_{ZC} or V_{SC}, to transfer energy, preferably heat, from at least a portion of a heat transfer medium selected from W*₃, W*₄ to the crude product RP_{C}.

In addition, it is also possible to direct the bottom product stream S_{cp} partly through a reboiler V_{SC} and then to recycle it partly back into RR_{C}, in which case, in V_{SC}, energy, preferably heat, is transferred from at least a portion of a heat transfer medium selected from W*₃, W*₄ to the recycled portion of S_{CP} and then, in column RR_{C}, from S_{CP} to crude product RP_{C} present in the column.

Energy is transferred here directly or indirectly from at least a portion of a heat transfer medium selected from W*₃, W*₄ to the streams mentioned, i.e. without or with heat transfer medium [symbol] , as described correspondingly in section 4.7.

The preferred embodiment of the process according to the invention makes it possible to efficiently use the energy from W*₃, W*₄, especially from W*₄, W*₃₁, W*₃₂. This reduces the total energy demand.

### 5. EXAMPLES

### 5.1 Example 1 (Noninventive), Corresponding to FIG. 1

The noninventive example corresponds to FIG. 1, except that the intermediate cooler WT_{X} <402> shown in FIG. 1 (or FIG. 2 or FIG. 3) is not utilized. This also applies to the noninventive Example 2, and to the inventive Example 3.

A stream of aqueous NaOH (50% by weight) S_{AE2} <102> of 5000 kg/h is fed at 30° C. to the top of a reaction column RR_{A} <100>. A vaporous methanol stream S_{AE1} <103> of 56700 kg/h is fed in countercurrent at the bottom of the reaction column RR_{A} <100>. The reaction column RR_{A} <100> is operated at a top pressure of 1.25 bar abs. At the bottom of the column RR_{A} <100>, a virtually water-free product stream S_{AP*} <104> of 11 000 kg/h is withdrawn (30% by weight sodium methoxide in methanol). The evaporator V_{SA} <105> of the reaction column RR_{A} <100> introduces about 1200 KW of heating power using low pressure steam. A vaporous methanol-water stream S_{AB} <107> is withdrawn at the top of the reaction column RR_{A} <100> and 4000 kg/h thereof are condensed in the condenser K_{RRA} <108> and returned to the reaction column RR_{A} <100> as reflux while the remaining stream of 50800 kg/h is supplied to a rectification column RD_{A} <300>.

The rectification column RD_{A} <300> is operated at a top pressure of 1.1 bar abs. At the bottom of the rectification column RD_{A} <300>, a liquid water stream S_{UA} <304> of 3500 kg/h is discharged (500 ppmw of methanol). The bottom temperature of RD_{A} <300> is 105° C. at 1.2 bar abs. At the top of the rectification column RD_{A} <300>, a vaporous methanol stream S_{OA} <302> (1.1 bar, 67° C.; 200 ppmw of water) of 100 000 kg/h is withdrawn, of which 43 300 kg/h is used as reflux and directed through a condenser K_{RD} <407> in which most of the heat of condensation (9.4 MW) is utilized in order to evaporate the working medium W*₁ <701> n-butane at 61.8° C. and 6.7 bar abs. to give stream W*₂ <702>. The remaining vapour stream from RD_{A} <300> of 56 700 kg/h is fed to a compressor V_{DAB2} <303>, compressed to 1.7 bar abs. therein and recycled to the reaction column RR_{A} <100>.

The gaseous n-butane stream W*₂ <702> is fed to a compressor VD₁ <401>, and preferably heated upstream of that through a superheater (ΔT=20 K). Subsequently, the resultant stream W*₃ <703> is fed to the compressor VDₓ <405>, such that there is multistage compression of stream W*₂ <702> to give stream W*₄ <704>. A total of 169 t/h of W*₂ <702> is compressed to give stream W*₄ <704> (18.6 bar abs.). This corresponds to a condensation temperature of 110.5° C. In the heat transferrer V_{SRD} <406>, a temperature differential of 5 K is established, and the heat is transferred from W*₄ <704> to a portion S_{UA1} <320> of the vapour stream S_{UA} <304>, so as again to result in a stream W*₁ <701> that can be fed again to the condenser K_{RD} <407>.

Before stream W*₁ <701> is fed again to the condenser K_{RD} <407>, it is possible to utilize any residual heat present in the condensed stream W*₁ <701> for superheating of the n-butane stream upstream of the compressor VD₁ <401>.

A total electrical compressor output of 2.7 MW is required, while no external heating media (e.g. steam) were required.

### 5.2 Example 2 (Noninventive), Corresponding to FIG. 2

The arrangement in the noninventive Example 2 corresponds to that of Example 1 with the following differences:

The rectification column RD_{A} <300> comprises an intermediate evaporator V_{ZRD} <409>. A liquid stream S_{ZA} <305> is withdrawn from the rectification column RD_{A} <300> at 80° C. and about 10 500 KW of heat is transferred thereto in the intermediate evaporator V_{ZRD} <409>, with partial evaporation of the stream, which is then returned to the rectification column RD_{A} <300>. At the top of the rectification column RD_{A} <300>, a vaporous methanol stream S_{OA} <302> (1.1 bar, 67° C.; 200 ppm by weight of water) of 100 000 kg/h is withdrawn, of which 43 300 kg/h is used as reflux and directed through a condenser K_{RD} <407> in which most of the heat of condensation (9.5 MW) is utilized in order to evaporate the working medium W*₁ <701> n-butane at 61.8° C. and 6.7 bar abs. to give stream W*₂ <702>. The remaining vapour stream of 56 700 kg/h is fed to a compressor V_{DAB2} <303>, where it is compressed to 1.7 bar abs., and returned to the reaction column RR_{A} <100>.

The gaseous n-butane stream W*₂ <702> is fed to a compressor VD₁ <401>, and preferably heated upstream of that through a superheater (ΔT=13 K). The stream W*₃ <703> is obtained. A total of 127 t/h of W*₂ <702> is compressed to give stream W*₃ <703> (11.5 bar abs.).

By comparison with Example 1, the pressure level of the n-butane working fluid does not have to be selected at such a high level, since the temperature in the intermediate evaporator V_{ZRD} <409> is 80° C. (and not 105° C. as in the bottom of RD_{A} <300>). Accordingly, the electrical power of the compressor VD₁ <401> is reduced compared to Example 1. Because of the smaller temperature jump, the power of the compressor is 1 MW. A thermal power for V_{ZRD} <409> of 10.5 MW is provided. Nevertheless, the use of a heat pump in the form described cannot achieve complete electrification of the RD_{A} <300>. The reboiler <406> is operated with low-pressure steam (alternatively: a different waste heat source), and it is necessary to use 2.04 MW. Accordingly, a total of 1.0 MW of electrical power and 2.04 MW of heating steam are used.

### 5.3 Example 3 (Inventive), Corresponding to FIG. 3

The arrangement in the inventive Example 3 corresponds to that of Examples 1 and 2 with the following differences:

A vaporous methanol stream S_{OA} <302> (1.1 bar, 67° C.; 200 ppmw of water) of 100 000 kg/h is withdrawn at the top of the rectification column RD_{A} <300>, of which 43 300 kg/h are used as reflux and directed through a condenser K_{RD} <407>.

11.14 MW of heat of condensation is used in the condenser K_{RD} <407> in order to evaporate the working medium W*₁ <701> n-butane at 61.8° C. and 6.7 bar abs. to give stream W*₂ <702>. The remaining vapour stream from RD_{A} <300> of 56 700 kg/h is fed to a compressor V_{DAB2} <303>, compressed therein to 1.7 bar abs. and recycled to the reaction column RR_{A} <100>.

The gaseous n-butane stream W*₂ <702> is fed to a compressor VD₁ <401>, and preferably upstream of that through a superheater (ΔT=20 K). Subsequently, the resultant stream W*_{3 <703}> (11.5 bar, 110.5° C.) is divided.

127 t/h of n-butane from stream W*₃ <703> (=stream W*₃₁ <7031>) are directed at 11.5 bar abs. to the side evaporator V_{ZRD} <409> in order to transmit 10.5 MW there.

The remaining portion of stream W*₃ <703>, i.e. W*₃₂ <7032>, 29 t/h, is fed to the compressor VDₓ <405> and compressed from 11.5 bar abs. to 18.6 bar abs. to give stream W*₄ <704>. Stream W*₄ <704 > is then condensed in the reboiler V_{SRD} <406> (2.04 MW).

A total of 1.4 MW of electrical power is required for the compressor stages VD₁ <401> and VD₂ <405>.

By comparison with Example 1 and Example 2, the total energy requirement is reduced with establishment of the same boundary conditions and the same power. Moreover, compared to Example 2, no additional heating medium is used. This column is thus fully electrified. In the case of use of green power, it is thus possible to assure CO₂-free separation.

The total energy to be supplied is minimized by the process according to Example 3.

The proportion of heating power provided by low pressure steam and compressor power required in each case is shown in FIG. 10.

Result: The inventive procedure of staged compression of the heat transfer medium and therefore of operating the intermediate evaporator and reboiler with the heat transfer medium compressed to different extents can surprisingly achieve energy savings.

1-15. (canceled)

16. A process for preparing at least one alkali metal methoxide of the formula M_{A}OCH₃ wherein M_{A} is sodium, lithium, or potassium, said process comprising:
(a1) a reactant stream S_{AE1} comprising methanol is reacted with a reactant stream S_{AE2} comprising M_{A}OH in countercurrent in a reactive rectification column RR_{A} to produce a crude product RP_{A} comprising M_{A}OCH₃, water, methanol, M_{A}OH;
wherein a bottom product stream S_{AP} comprising methanol and M_{A}OCH₃ is withdrawn at the lower end of RR_{A} and a vapour stream S_{AB} comprising water and methanol is withdrawn at the upper end of RR_{A};
(a2) simultaneously with, and spatially separate from, step (a1), a reactant stream S_{BE1} comprising methanol is reacted with a reactant stream S_{BE2} comprising M_{B}OH in countercurrent in a reactive rectification column RR_{B} to give a crude product RP_{B} comprising M_{B}OCH₃, water, methanol, and M_{B}OH, wherein M_{B} is selected from sodium, lithium, potassium;
wherein a bottom product stream S_{BP} comprising methanol and M_{B}OCH₃ is withdrawn at the lower end of RR_{B} and a vapour stream S_{BB} comprising water and methanol is withdrawn at the upper end of RR_{B};
(a3) at least a portion of vapour stream S_{AB}, and at least a portion of vapour stream S_{BB}, mixed with S_{AB} or separate from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A};
(b) at least one side stream S_{ZA} is withdrawn from RD_{A} and recycled back into RD_{A};
(c) energy is transferred from at least a portion of S_{OA} to a liquid or gaseous heat transfer medium W*₁, to produce gaseous heat transfer medium W*₂;
(d) at least a portion of the gaseous heat transfer medium W*₂ is compressed to obtain a gaseous heat transfer medium W*₃ that has been compressed relative to W*₂;
(e) energy is transferred from a first portion W*₃₁ of the gaseous heat transfer medium W*₃ to S_{ZA} before S_{ZA} is recycled into RD_{A};
(f) a portion of the gaseous heat transfer medium W*₃ other than W*₃₁, W*₃₂, is compressed further to obtain a gaseous heat transfer medium W*₄ that has been compressed relative to W*₃₁; and
(g) energy is transferred from at least a portion of W*₄ to at least a portion S_{UA1} of S_{UA} before S_{UA1} is recycled into RD_{A}.

17. The process of claim 16, wherein, in step (e), energy is transferred from W*₃₁ to S_{ZA} in an intermediate evaporator V_{ZRD}.

18. The process of claim 16, wherein, in step (g), energy is transferred from at least a portion of W*₄ to the at least one portion S_{UA1} of S_{UA} in a reboiler V_{SRD}.

19. The process of claim 16, wherein, once energy has been transferred from W*₃₁ to S_{ZA} as per step (e), energy is transferred from at least a portion of W*₃₁ to S_{OA}, and/or once energy has been transferred from at least a portion of W*₄ to S_{UA1} as per step (g), energy is transferred from at least a portion of W*₄ to S_{OA}.

20. The process of claim 16, wherein at least two of the columns selected from rectification column RD_{A}, reaction column RR_{A} and reaction column RR_{B} are accommodated in one column shell, wherein the columns are at least partially separated from one another by a dividing wall extending to the bottom of the column.

21. The process of claim 16, wherein a portion of S_{OA} is used as reactant stream S_{AE1} in step (a1), and alternatively or in addition, as reactant stream S_{BE1} in step (a2).

22. The process of claim 16, wherein energy is transferred from at least a portion of a stream selected from W*₃, or W*₄ to the crude product RP_{A} and, alternatively or in addition to, crude product RP_{B}.

23. The process of claim 16, wherein M_{A} is sodium or potassium, and M_{B} is sodium or potassium.

24. The process of claim 23, wherein M_{A} is sodium, and M_{B} is potassium.

25. The process of claim 16, wherein:
in a reactive rectification column RR_{C}, a reactant stream S_{CE1} comprising M_{c}OR′ is reacted in countercurrent with a reactant stream S_{CE2} comprising R″OH to produce a crude product RP_{C} comprising M_{C}OR″ and R′OH;
wherein a bottom product stream S_{CP} comprising M_{C}OR″ is withdrawn at the lower end of RR_{C} and a vapour stream S_{CB} comprising R′OH is withdrawn at the upper end of RR_{C};
R′ and R″ are two different C₁ to C₆ hydrocarbon radicals, and M_{c} is a metal selected from lithium, sodium, potassium; and
energy is transferred from at least a portion of a stream selected from W*₃, or W*₄ to the crude product RP_{C}.

26. The process of claim 25, wherein R′=methyl.

27. The process of claim 26, wherein at least a portion of S_{AP} is used as S_{CE1}.

28. The process of claim 26, wherein at least a portion of S_{BP} is used as S_{CE1}.

29. The process of claim 26 wherein R″ is selected from the group consisting of: ethyl, n-propyl, iso-propyl, sec-butyl, 2-methyl-2-butyl, tert-butyl, 2-methyl-2-pentyl, 3-methyl-3-pentyl, 3-ethyl-3-pentyl, 2-methyl-2-hexyl, and 3-methyl-3-hexyl.

30. The process of claim 29, wherein R″=ethyl.

31. A process for preparing at least one alkali metal methoxide of the formula M_{A}OCH₃ wherein M_{A} is selected sodium, lithium, or potassium, and wherein:
(a1) a reactant stream S_{AE1} comprising methanol is reacted with a reactant stream S_{AE2} comprising M_{A}OH in countercurrent in a reactive rectification column RR_{A} to produce a crude product RP_{A} comprising M_{A}OCH₃, water, methanol, and M_{A}OH;
wherein a bottom product stream S_{AP} comprising methanol and M_{A}OCH₃ is withdrawn at the lower end of RR_{A} and a vapour stream S_{AB} comprising water and methanol is withdrawn at the upper end of RR_{A};
(a3) at least a portion of the vapour stream S_{AB}, and at least a portion of the vapour stream S_{BB}, mixed with S_{AB} or separate from S_{AB}, is directed into a rectification column RD_{A} and separated in RD_{A} into at least one vapour stream S_{OA} comprising methanol, which is withdrawn at the upper end of RD_{A}, and at least one stream S_{UA} comprising water, which is withdrawn at the lower end of RD_{A};
(b) at least one side stream S_{ZA} is withdrawn from RD_{A} and recycled back into RD_{A};
(c) energy is transferred from at least a portion of S_{OA} to a liquid or gaseous heat transfer medium W*₁, to produce a gaseous heat transfer medium W*₂;
(d) at least a portion of the gaseous heat transfer medium W*₂ is compressed to obtain a gaseous heat transfer medium W*₃ that has been compressed relative to W*₂;
(e) energy is transferred from a first portion W*₃₁ of the gaseous heat transfer medium W*₃ to S_{ZA} before S_{ZA} is recycled into RD_{A};
(f) a portion of the gaseous heat transfer medium W*₃ other than W*₃₁, W*₃₂, is compressed further to obtain a gaseous heat transfer medium W*₄ that has been compressed relative to W*₃₁; and
(g) energy is transferred from at least a portion of W*₄ to at least a portion S_{UA1} of S_{UA} before S_{UA1} is recycled into RD_{A}.

32. The process of claim 31, wherein M_{A} is selected from sodium or potassium.

33. The process of claim 31, wherein:
in a reactive rectification column RR_{C} a reactant stream S_{CE1} comprising M_{c}OR′ is reacted in countercurrent with a reactant stream S_{CE2} comprising R″OH to produce a crude product RP_{C} comprising M_{C}OR″ and R′OH;
wherein a bottom product stream S_{CP} comprising M_{C}OR″ is withdrawn at the lower end of RR_{C} and a vapour stream S_{CB} comprising R′OH is withdrawn at the upper end of RR_{C};
R′ and R″ are two different C₁ to C₆ hydrocarbon radicals, and M_{C} is a metal selected from lithium, sodium, potassium; and
energy is transferred from at least a portion of a stream selected from W*₃, W*₄ to the crude product RP_{C}.

34. The process of claim 31, wherein R′=methyl.

35. The process of claim 34, wherein R″ is selected from the group consisting of: ethyl; n-propyl; iso-propyl; sec-butyl; 2-methyl-2-butyl; tert-butyl; 2-methyl-2-pentyl; 3-methyl-3-pentyl; 3-ethyl-3-pentyl; 2-methyl-2-hexyl; and 3-methyl-3-hexyl.
